(19) United States
(12) Patent Application Publication
(10) Pub. No.: US 2005/0054908 A1
(43) Pub. Date: Mar. 10, 2005

(54) Photostimulation method and apparatus in combination with glucose determination

(76) Inventors: Thomas B. Blank, Chandler, AZ (US); Stephen L. Monfre, Gilbert, AZ (US); Marcy Makarewicz, Chandler, AZ (US); Mutua Mattu, Gilbert, AZ (US); Kevin H. Hazen, Gilbert, AZ (US); James R. Henderson, Phoenix, AZ (US)

Correspondence Address: GLENN PATENT GROUP, 3475 EDISON WAY, SUITE L, MENLO PARK, CA, 94025, US

(21) Appl. No.: 10/841,200

(22) Filed: May 6, 2004

Related U.S. Application Data
(60) Provisional application No. 60/504,099, filed on Sep. 19, 2003.
(60) Provisional application No. 60/472,613, filed on May 21, 2003.

(30) Foreign Application Priority Data
Mar. 7, 2003 (WO) PCT/US03/07065

(51) Int. Cl. A61B 5/00; A61B 6/00

(52) U.S. Cl. 600/316; 600/473; 600/476; 600/407

(57) ABSTRACT

A method and apparatus using photo-stimulation to treat or pretreat a sample site prior to analyte concentration determination is presented. More particularly, photo-stimulation at or near at least one sample site is used to enhance perfusion of the sample site leading to reduced errors associated with sampling. Increased perfusion of the sample site leads to increased volume percentages of the target analyte and/or allows the blood or tissue constituent concentrations to more accurately and/or precisely track corresponding sample constituents in more well perfused body compartments or sites such as arteries, veins, or fingertips. In one embodiment, analysis of the photo-stimulated site is used in conjunction with glucose analyzers to determine the analyte concentration with greater ease, accuracy, or precision and may allow determination of the analyte concentration of another non-sampled body part or compartment.

### CROSS REFERENCE TO RELATED APPLICATIONS

This document claims priority to PCT patent application No. PCT/US03/07065, filed Mar. 7, 2003, which claims priority to U.S. provisional patent application No. 60/362,885, filed Mar. 8, 2002 and U.S. provisional patent application No. 60/448,840, filed Feb. 19, 2003 (Attorney docket number SENS0011), U.S. provisional patent application No. 60/504,099, Sep. 19, 2003. (Attorney docket number SENS0034PR), and U.S. provisional application No. 60/472,613, filed May 21, 2003 (Attorney docket number SENS0022), all of which are incorporated herein in their entirety by this reference thereto.

### BACKGROUND OF THE INVENTION

1. Technical Field

The invention relates generally to biomedical methods and apparatus. More particularly, the invention relates to preparing a tissue sample site for analysis. Still more particularly, the invention relates to the use of photonic stimulation to enhance perfusion of glucose concentrations between body fluid compartments in combination with glucose sampling and/or glucose analysis techniques.

2. Description of the Prior Art

### Diabetes

Diabetes is a chronic disease that results in improper production and use of insulin, a hormone that facilitates glucose uptake into cells. While a precise cause of diabetes is unknown, genetic factors, environmental factors, and obesity appear to play roles. Diabetics have increased risk in three broad categories: cardiovascular heart disease, retinopathy, and neuropathy. Complications of diabetes include: heart disease and stroke, high blood pressure, kidney disease, neuropathy (nerve disease and amputations), retinopathy, diabetic ketoacidosis, skin conditions, gum disease, impotence, and fetal complications. Diabetes is a leading cause of death and disability worldwide. Moreover, diabetes is merely one among a group of disorders of glucose metabolism that also includes impaired glucose tolerance, and hyperinsulinemia, or hypoglycemia.

Diabetes Prevalence and Trends

Diabetes is an ever more common disease. The World Health Organization (WHO) estimates that diabetes currently afflicts 154 million people worldwide. There are 54 million people with diabetes living in developed countries. The WHO estimates that the number of people with diabetes will grow to 300 million by the year 2025. In the United States, 15.7 million people or 5.9 per cent of the population are estimated to have diabetes. Within the United States, the prevalence of adults diagnosed with diabetes increased by six percent in 1999 and rose by 33 percent between 1990 and 1998. This corresponds to approximately eight hundred thousand new cases every year in America. The estimated total cost to the United States economy alone exceeds $90 billion per year. See Diabetes Statistics, National Institutes of Health, Publication No. 98-3926, Bethesda, Md. (November 1997).

Diabetes Detection and Management

Diagnosis of diabetes is traditionally performed in a professional setting. These diagnosis are often performed with glucose or meal tolerance tests followed by one or more glucose determinations over a period time ranging from about one to four hours. Diagnostic tests are performed with a number of invasive or minimally invasive techniques. Noninvasive techniques are also being developed for this purpose.

Once diagnosed, long-term clinical studies demonstrate that the onset of diabetes related complications is significantly reduced through proper control of blood glucose concentrations. See The Diabetes Control and Complications Trial Research Group, The effect of intensive treatment of diabetes on the development and progression of long-term complications in insulin-dependent diabetes mellitus, N Eng J of Med, 329:977-86 (1993). Long term control of glucose concentrations of non-insulin dependent diabetics has also been shown to reduce diabetes related complications. See U.K. Prospective Diabetes Study (UKPDS) Group, Intensive blood-glucose control with sulphonylureas or insulin compared with conventional treatment and risk of complications in patients with type 2 diabetes, Lancet, 352:837-853 (1998); and Y. Ohkubo, H. Kishikawa, E. Araki, T. Miyata, S. Isami, S. Motoyoshi, Y. Kojima, N. Furuyoshi, M. Shichizi, Intensive insulin therapy prevents the progression of diabetic microvascular complications in Japanese patients with non-insulin-dependent diabetes mellitus: a randomized prospective 6-year study, Diabetes Res Clin Pract, 28:103-117 (1995). More recently, studies have indicated that testing and control of pre-diabetics leads to a significant delay of the onset of diabetes related complications.

### Glucose Measurement History, Approaches, and Technologies

The treatment of diabetes has progressed through several stages. The combined development- of insulin therapy and in-home glucose determination led to a radical improvement in the lives of diabetics. Home glucose determination has progressed through multiple stages. Urine tests for glucose have given way to the invasive fingerstick glucose determinations that are more accurate but somewhat painful. The development of alternative site glucose concentration determinations has somewhat mitigated the pain aspects, but maintains a biohazard issue and may have introduced a difficulty in temporal and spatial differences in glucose concentration between the well perfused fingertip and the less well perfused alternative sites. Current research is focusing on the development of noninvasive technologies that will totally eliminate the pain associated with glucose concentration determination and fluid biohazard issues. Finally, considerable progress has been made in implantable or full-loop systems incorporating both glucose concentration determination and insulin delivery that will result in the realization of an artificial pancreas.

Blood glucose concentration determination is categorized into four major types: traditional invasive, alternative invasive, noninvasive, and implantable. Due to the wide use of these modes of measurement and somewhat loose use of terminology in the literature, a detailed summary of the terminology for each mode of measurement is provided here to clarify usage within this document.

In the medical field, invasive often refers to surgery. That is not the definition of invasive herein. In the glucose concentration determination field, invasive is a term defined relative to noninvasive. Noninvasive is a method in which no biological sample or fluid is taken from the body to perform a glucose measurement. Invasive then means that a biological sample is collected from the body. Invasive glucose concentration determinations is further separatedinto two groups. The first is a traditional invasive method in which a blood sample is collected from the body from an artery, vein, or capillary bed in the fingertips or toes. The second is an alternative invasive method in which a blood, interstitial fluid, or biological fluid sample is drawn from a region other than an artery, vein, or capillary bed in the fingertips or toes. A further description of these terms is provided in the remainder of this section.

Traditional Invasive Glucose Determination

There are three major categories of traditional (classic) invasive glucose determinations. The first two methodologies use blood drawn with a needle from an artery or vein, respectively. The third methodology uses capillary blood obtained via lancet from the fingertip or toes. Over the past two decades, this has become the most common method for self-monitoring of blood glucose at home, at work, or in public settings.

Common technologies are used to analyze the blood collected by venous draw and finger stick approaches. Glucose concentration analysis include techniques such as calorimetric and enzymatic glucose analysis. The most common enzymatic based glucose analyzers use glucose oxidase, which catalyzes the reaction of glucose with oxygen to form gluconolactone and hydrogen peroxide, see Equation (1) below. Glucose concentration determination include techniques based upon depletion of oxygen in the sample, that use the changes in sample pH, or that use the formation of hydrogen peroxide. A number of calorimetric and electro enzymatic techniques further use the reaction products as a starting reagent. For example, hydrogen peroxide reacts in the presence of platinum to form the hydrogen ion, oxygen, and current any of which is used indirectly to determine the glucose concentration, see Equation (2) below.
glucose+O₂→gluconolactone+H₂O₂ (1)
H₂O₂→2H⁺O₂+2e⁻ (2)

It is noted that a number of alternative site methodologies such as the TheraSense® FreeStyle™ collect blood samples from regions other than the fingertip or toes. These technologies are not herein referred to as traditional invasive glucose meters unless the sample is drawn from the fingertip or toes despite having similar chemical analyses such as the calorimetric or enzymatic analysis described above. To further clarify, a TheraSense® FreeStyle™ meter used to collect blood via lancet from sample sites consisting of the fingertip or toe is a traditional invasive glucose analyzer.

Alternative Invasive Glucose Determination

There are several alternative invasive methods of determining glucose concentration.

A first group of alternative invasive glucose concentration analyzers have a number of similarities to the traditional invasive glucose concentration analyzers. One similarity is that blood samples are acquired with a lancet. This form of alternative invasive glucose determination is not used to collect for analysis venous or arterial blood but is used to collect capillary blood samples. A second similarity is that the blood sample is analyzed using chemical analyses that are similar to the colorimetric and enzymatic analyses describe above. The primary difference is that in an alternative invasive glucose determination the blood sample is not collected from the fingertip or toes. For example, according to package labeling the TheraSense® FreeStyle Meter™ may be used to collect and analyze blood from the forearm. This is an alternative invasive glucose determination due to the location of the lancet draw. In this first group of alternative invasive methods based upon blood draws with a lancet, a primary difference between the alternative invasive and traditional invasive glucose determination is the location of blood acquisition from the body. Additional differences include factors such as the gauge of the lancet, the depth of penetration of the lancet, timing issues, the volume of blood acquired, and environmental factors such as the partial pressure of oxygen, altitude, and temperature. This form of alternative invasive glucose determination comprises samples collected from the palmar region, base of thumb, forearm, upper arm, head, earlobe, torso, abdominal region, thigh, calf, and plantar region.

A second group of alternative invasive glucose analyzers are distinguished by their mode of sample acquisition. This group of glucose analyzers has a common characteristic of acquiring a biological sample from the body or modifying the surface of the skin to gather a sample without use of a lancet for subsequent analysis. For example, a laser poration based glucose concentration analyzer would use a burst or stream of photons to create a small hole in the surface of the skin. A sample of basically interstitial fluid collects in the resulting hole. Subsequent analysis of the sample for glucose constitutes an alternative invasive glucose concentration analysis whether or not the sample is actually removed from the created hole. A second common characteristic is that a device and algorithm are used to determine glucose concentration from the sample. Herein, the term alternative invasive include techniques that analyze biosamples such as interstitial fluid, whole blood, mixtures of interstitial fluid and whole blood, and selectively sampled interstitial fluid. An example of selectively sampled interstitial fluid includes collected fluid in which large or less mobile constituents are not fully represented in the resulting sample. For this second group of alternative invasive glucose analyzers sampling sites include: the hand, fingertips, palmar region, base of thumb, forearm; upper arm, head, earlobe, eye, chest, torso, abdominal region, thigh, calf, foot, plantar region, and toes. A number of methodologies exist for the collection of the sample for alternatively invasive measurements including laser poration, applied current, and suction. The most common are summarized here:
A. Laser poration: In these systems, photons of one or more wavelengths are applied to skin creating a small hole in the skin barrier. This allows small volumes of interstitial fluid to become available to a number of sampling techniques.
B. Applied current: In these systems, a small electrical current is applied to the skin allowing interstitial fluid to permeate through the skin.
C. Suction: In these systems, a partial vacuum is applied to a local area on the surface of the skin. Interstitial fluid permeates the skin and is collected.

In all of these techniques, the analyzed sample is interstitial fluid. However, some of the techniques are applied to the skin in a fashion that draws blood. For example, the laser poration method results in biological fluid droplets. In this document, any technique that draws biosamples from the skin without the use of a lancet on the fingertip or toes is referred to as alternative invasive technique. In addition, it is recognized that the alternative invasive systems each have different sampling approaches that lead to different subsets of the interstitial fluid being collected. For example, large proteins might lag behind in the skin while smaller, more diffusive, elements are preferentially sampled. This leads to samples being collected with varying analyte and interferent concentrations. Another example is that a mixture of whole blood and interstitial fluid is collected. These techniques are optionally used in combination. For example the Soft-Tact, SoftSense in Europe, applies suction to the skin followed by a lancet stick. Despite the differences in sampling, these techniques are referred to as alternative invasive techniques sampling interstitial fluid.

Sometimes, the literature refers to the alternative invasive technique as an alternative site glucose determination or as a minimally invasive technique. The minimally invasive nomenclature derives from the method by which the sample is collected. In this document, the alternative site glucose concentration determinations that draw blood or interstitial fluid, even ¼ microliter, are considered to be alternative invasive glucose concentration determination techniques as defined above. Examples of alternative invasive techniques include the TheraSense® FreeStyle™ when not sampling fingertips or toes, the Cygnus® GlucoWatch™, the One Touch® Ultra™, and equivalent technologies.

Biosamples collected with alternative invasive techniques are analyzed via a large range of technologies. The most common of these technologies are summarized below:
A. Conventional: With some modification, the interstitial fluid samples are analyzed by most of the technologies used to determine glucose concentrations in serum, plasma, or whole blood. These include electrochemical, electroenzymatic, and calorimetric approaches. For example, the enzymatic and colorimetric approaches described above are also used to determine the glucose concentration in interstitial fluid samples.
B. Spectrophotometric: A number of approaches, for determining the glucose concentration in biosamples, have been developed that are based upon spectrophotometric technologies. These techniques include: Raman and fluorescence, as well as techniques using light from the ultraviolet through the infrared [ultraviolet (200 to 400 nm), visible (400 to 700 nm), near-infrared (700 to 2500 nm or 14,286 to 4000 cm⁻¹), and infrared (2500 to 14,285 nm or 4000 to 700 cm⁻¹)].

In this document, an invasive glucose concentration analyzer is the genus of both a traditional invasive glucose analyzer species and an alternative invasive glucose analyzer species.

Noninvasive Glucose Determination

There exist a number of noninvasive approaches for glucose concentration determination. These approaches vary widely, but have at least two common steps. First, an apparatus is used to acquire a reading from the body without obtaining a biological sample. Second, an algorithm is used to convert this reading into a glucose determination.

One species of noninvasive glucose concentration analyzer are those based upon spectra. Typically, a noninvasive apparatus uses some form of spectroscopy to acquire the signal or spectrum from the body. Used spectroscopic techniques include but are not limited to Raman, fluorescence, as well as techniques using light from ultraviolet through the infrared [ultraviolet (200-400 nm), visible (400-700 nm), near-IR (700 to 2500 nm or 14,286 to 4000 cm⁻¹), and infrared (2500 to 14,285 nm or 4000-700 cm⁻¹)]. A particular range for noninvasive glucose determination in diffuse reflectance mode is about 1100 to 2500 nm or ranges therein. See K. Hazen, Glucose Determination in Biological Matrices Using Near-Infrared Spectroscopy, doctoral dissertation, University of Iowa, 1995. It is important to note, that these techniques are distinct from the traditionally invasive and alternative invasive techniques listed above in that the sample analyzed is a portion of the human body in-situ, not a biological sample acquired from the human body.

Typically, one or more of three modes are used to collect noninvasive scans: transmittance, transflectance, and diffuse reflectance. For example the light, spectrum, or signal collected is light transmitted through a region of the body such as a fingertip, diffusely reflected, or transflected. Transflected here refers to collection of the signal not at the incident point or area (diffuse reflectance), and not at the opposite side of the sample (transmittance), but rather at some point on the body between the transmitted and diffuse reflectance collection area. For example, transflected light enters the fingertip or forearm in one region and exits in another region typically 0.2 to 5 mm or more away depending on the wavelength used. For example, light that is strongly absorbed by the body such as light near water absorbance maxima at 1450 or 1950 nm must be collected after a small radial divergence and light that is less absorbed such as light near water absorbance minima at 1300, 1600, or 2250 nm may be collected at greater radial or transflected distances from the incident photons.

Noninvasive techniques are not limited to the fingertip. Other regions or volumes of the body subjected to noninvasive measurements include: a hand, finger, palmar region, base of thumb, back of wrist, forearm, volar aspect of the forearm, dorsal aspect of the forearm, upper arm, head, earlobe, eye, tongue, chest, torso, abdominal region, thigh, calf, foot, plantar region, and toe. It is important to note that noninvasive techniques do not have to be based upon spectroscopy. For example, a bioimpedence meter is considered to be a noninvasive device. In this document, any device that reads glucose from the body without penetrating the skin and collecting a biological sample is referred to as a noninvasive glucose analyzer. For the purposes of this document. X-rays and magnetic resonance images (MRI's) are not considered to be defined in the realm of noninvasive technologies.

Implantable Sensor for Glucose Determination

There exist a number of approaches for implanting a glucose sensor into the body for glucose determination. These implantables are used to collect a sample for further analysis or are used to acquire a reading of the sample directly or indirectly. Two categories of implantable glucose analyzers exist: short-term and long-term.

In this document, a device or a collection apparatus is referred to as at least a short-term implantable, as opposed to a long-term implantable, if part of the device penetrates the skin for a period of greater than three hours and less than one month. For example, a wick placed subcutaneously to collect a sample overnight that is removed and analyzed for glucose content representative of the interstitial fluid glucose concentration is referred to as a short term implantable. Similarly, a biosensor or electrode placed under the skin for a period of greater than three hours that reads directly or indirectly a glucose concentration is referred to as at least a short-term implantable device. Conversely, devices described above based upon techniques such as a lancet, applied current, laser poration, or suction are referred to as either a traditional invasive or alternative invasive technique as they do not fulfill both the three hour and penetration of skin parameters. In this document, long-term implantables are distinguished from short-term implantables by having the criteria that they must both penetrate the skin and be used for a period of one month or longer. Long term implantables may be in the body for one or many years.

Implantable glucose concentration analyzers vary widely, but include at least three common steps. First, at least part of the device penetrates the skin. More commonly, the entire device is imbedded into the body. Second, the apparatus is used to acquire either a sample of the body or a signal relating directly or indirectly to the glucose concentration within the body. If the implantable device collects a sample, readings or measurements on the sample are collected after removal from the body. Alternatively, readings are transmitted out of the body by the device or used for such purposes as insulin delivery while in the body. Third, an algorithm is used to convert the signal into a reading directly or indirectly related to the glucose concentration. An implantable analyzer samples one or more of a variety of body fluids or tissues including arterial blood, venous blood, capillary blood, interstitial fluid, and selectively sampled interstitial fluid. An implantable analyzer may also collect glucose information from skin tissue, cerebral spinal fluid, organ tissue, or through an artery or vein. For example, a implantable glucose analyzer is placed transcutaneously, in the peritoneal cavity, in an artery, in muscle, or in an organ such as the liver or brain. The implantable glucose sensor is one component of an artificial pancreas.

Examples of implantable glucose monitors follow. One example of a Continuous Glucose Monitoring System (CGMS) is a group of glucose monitors based upon open-flow microperfusion. See Z. Trajanowski, G. Brunner, L. Schaupp, M. Ellmerer, P. Wach, T. Pieber, P. Kotanko, F. Skrabai, Open-Flow Microperfusion of Subcutaneous Adipose Tissue for ON-Line Continuous Ex Vivo Measurement of Glucose Concentration, Diabetes Care, 20, 1997, 1114-1120. Another example uses implanted sensors that comprise biosensors and amperometric sensors. See Z. Trajanowski, P. Wach, R. Gfrerer, Portable Device for Continuous Fractionated Blood Sampling and Continuous ex vivo Blood Glucose Monitoring, Biosensors and Bioelectronics, 11, 1996, 479-487. Another example is the MiniMed® CGMS.

### Glucose Distribution

A number of reports exist that indicate that the glucose concentration in alternative sites such as the forearm differ from those of traditional sample sites such as the fingertip. This area was previously described in U.S. application Ser. No. 10/377,916, which is incorporated herein in its entirety by this reference thereto.

Many papers claim that alternative site glucose concentrations are equivalent to fingerstick glucose determination. A number of examples are summarized in this section.

Szuts from Abbott Laboratories concluded that measurable physiological differences in glucose concentration between the arm and fingertip could be determined, but that these differences were found to be clinically insignificant even in those subjects in whom they were measured. See E. Szuts, P. Lock, K. Malomo, A. Anagnostopoulos, Blood Glucose Concentrations of Arm and Finger During Dynamic Glucose Conditions, Diabetes Technology & Therapeutics, 4, 3-11 (2002).

Lee from Roche Diagnostics Corporation concluded that patients testing two-hours Postprandial could expect to see small differences between their forearm and fingertip glucose concentrations. See D. Lee, S. Weinert, E. Miller, A Study of Forearm Versus Finger Stick Glucose Monitoring, Diabetes Technology & Therapeutics, 4, 13-23 (2002).

McGarraugh from TheraSense, Inc. concluded that there is no significant difference in HbA₁C measurements for patients using alternative site meters off of the fingertip and traditional glucose analyzers on the fingertip. See N. Bennion, N. Christensen, G. McGarraugh, Alternate Site Glucose Testing: A Crossover Design, Diabetes Technology & Therapeutics, 4, 25-33 (2002). This is an indirect indication that the forearm and fingertip glucose concentrations are the same, though many additional factors such as pain and frequency of testing will impact the study.

Peled from Amira Medical concluded that glucose concentration monitoring of blood samples from the forearm is suitable when expecting steady state glycemic conditions and that the palm samples produced a close correlation with fingertip glucose concentration determinations under all glycemic states. See N. Peled, D. Wong, S. Gwalani, Comparison of Glucose Levels in Capillary Blood Samples from a Variety of Body Sites, Diabetes Technology & Therapeutics, 4, 35-44 (2002).

Based upon a study using fast acting insulin injected intravenously, Koschinsky suggested that to avoid risky delays of hyperglycemia and hypoglycemia detection that monitoring at the arm should be limited to situations in which ongoing rapid changes in the blood glucose concentration can be excluded. See K. Jungheim, T. Koschinsky, Glucose Monitoring at the Arm, Diabetes Care, 25, 956-960 (2002) and K. Jungheim, T. Koschinsky, Risky Delay of Hypoglycemia Detection by Glucose Monitoring at the Arm, Diabetes Care, 24, 1303-1304 (2001). The use of intravenous insulin in this study was criticized as creating physiological extremes that influence the observed differences. See G. McGarraugh, Response to Jungheim and Koschinsky, Diabetes Care, 24, 1304-1306 (2001).

Equilibration Approaches

While there exist multiple reports that glucose concentrations are very similar when collected from the fingertip or alternative locations, a number of sampling approaches have been recommended to increase localized perfusion at the sample site to equilibrate the values just prior to sampling. Several of these approaches are summarized here:
1. Pressure: One sampling methodology requires rubbing or applying pressure to the sampling site to increase localized perfusion prior to obtaining a sample via lancet. An example of this is TheraSense's FreeStyle blood glucose concentration analyzer. See G. McGarraugh, S. Schwartz, R. Weinstein, Glucose Measurements Using Blood Extracted from the Forearm and the Finger, TheraSense, Inc., ART01022 Rev. C, 2001 and G. McGarraugh, D. Price, S. Schwartz, R. Weinstein, Physiological Influences on Off-Finger Glucose Testing, Diabetes Technology & Therapeutics, 3, 367-376 (2001).
2. Heating: Heat applied to the localized sample site has been proposed as a mechanism for equalizing the concentration between the vascular system and skin tissue. Application of heat is used to dilate the capillaries allowing more blood flow, which leads towards equalization of the venous and capillary glucose concentrations. Alternatively, vasodilating agents such as nicotinic acid, methyl nicotinamide, minoxidil, nitroglycerin, histamine, capsaicin, or menthol can be used to increase local blood flow. See M. Rohrscheib, C. Gardner, M. Robinson, Method and Apparatus for Noninvasive Blood Analyte Measurement with Fluid Compartment Equilibration, U.S. Pat. No. 6,240,306 (May 29, 2001).
3. Vacuum: Applying a partial vacuum to the skin at and around the sampling site prior to sample collection has also been used. A localized deformation in the skin allows superficial capillaries to fill more completely. See T. Ryan, A Study of the Epidermal Capillary Unit in Psoriasis, Dermatologica, 138, 459-472 (1969). For example, Abbott uses a vacuum device at one-half atmosphere that pulls the skin up 3.5 mm in their integrated device. Abbott maintains this deformation results in increased perfusion that equalizes the glucose concentration between the alternative site and the fingertip. See R. Ng, Presentation to the FDA at the Clinical Chemistry & Clinical Toxicology Devices Panel Meeting, Gaithersburg, Md. (Oct. 29, 2001).
   Calibration

Glucose analyzers require calibration. This is true for all types of glucose concentration analyzers such as traditional invasive, alternative invasive, noninvasive, and implantable analyzers. One fact associated with noninvasive glucose concentration analyzers is that they are secondary in nature, that is, they do not measure blood glucose concentrations directly. This means that a primary method is required to calibrate these devices to measure blood glucose concentrations properly. Many methods of calibration exist.

### Calibration of Noninvasive Glucose Meters

One noninvasive technology, near-infrared spectroscopy, provides the opportunity for both frequent and painless noninvasive measurement of glucose concentration. This approach involves the illumination of a spot on the body with near-infrared (NIR) electromagnetic radiation, light in the wavelength range 750 to 2500 nm. The light is partially absorbed and scattered, according to its interaction with the constituents of the tissue. The actual tissue volume that is sampled is the portion of irradiated tissue from which light is transflected or diffusely transmitted to the spectrometer detection system. With near-infrared spectroscopy, a mathematical relationship between an in-vivo near-infrared measurement and the actual blood glucose concentration needs to be developed. This is achieved through the collection of in-vivo NIR measurements with corresponding blood glucose concentrations that are obtained directly through the use of measurement tools such as the YSI, HemoCue, or any appropriate and accurate traditional invasive reference device.

For spectrophotometric based analyzers, there are several univariate and multivariate methods that are used to develop this mathematical relationship. However, the basic equation which is being solved is known as the Beer-Lambert Law. This law states that the strength of an absorbance/reflectance measurement is proportional to the concentration of the analyte which is being measured as in Equation (3) below,
A=εbC (3)
where A is the absorbance/reflectance measurement at a given wavelength of light, ε is the molar absorptivity associated with the molecule of interest at the same given wavelength, b is the distance that the light travels, and C is the concentration of the molecule of interest (glucose).

Chemometric calibrations techniques extract the glucose related signal from the measured spectrum through various methods of signal processing and calibration including one or more mathematical models. The models are developed through the process of calibration on the basis of an exemplary set of spectral measurements known as the calibration set and associated set of reference blood glucose concentrations based upon an analysis of fingertip capillary blood or venous blood. Common multivariate approaches requiring an exemplary reference glucose concentration vector for each sample spectrum in a calibration include partial least squares (PLS) and principal component regression (PCR). Many additional forms of calibration are well known in the art.

Because every method has error, it is beneficial for the primary device, which is used to measure blood glucose concentration, to be as accurate as possible in order to minimize the error that propagates through the developed mathematical relationship. While it appears intuitive that any U.S. FDA approved blood glucose monitor could be used, for accurate verification of the secondary method a monitor which has an accuracy of less than 5% is desirable. Meters with increased error such as 10% are acceptable, though the error of the device being calibrated may increase.

Although the above is well-understood, one aspect that is forgotten is that secondary methods require constant verification that they are providing consistent and accurate measurements when compared to the primary method. This means that a method for checking blood glucose concentrations directly and comparing those concentrations with the given secondary method must be developed. Such monitoring is manifested in quality assurance and quality control programs. Bias adjustments are often made to a calibration as are adjustments to a calibration. In some cases the most appropriate calibration is selected based upon these secondary methods. Sometimes this approach is known as validation.

The difference between alternative site glucose concentrations and traditional site glucose concentrations introduces errors associated with sampling into alternative site glucose analyzers.

Instrumentation

Noninvasive glucose concentration measurement using a near-infrared analyzer generally involves the illumination of a small region of the body with near-infrared electromagnetic radiation (light in the wavelength range 700 to 2500 nm). The light is partially absorbed and partially scattered according to its interaction with the constituents of the tissue prior to exiting the sample and being directed to a detector. The detected light contains quantitative information that corresponds to the known interaction of the incident light with components of the body tissue including water, fat, protein, and glucose.

A noninvasive glucose analyzer has one or more beam paths from a source to a detector. A number of light sources are available including a blackbody source, a tungsten-halogen source, one or more LED's, or one or more laser diodes. For multi-wavelength spectrometers a wavelength selection device is used or a series of optical filters is used for wavelength selection. Wavelength selection devices include one or more gratings, prisms, and wavelength selective filters. Alternatively, variation of the source such as varying which LED or diode is firing is used for wavelength selection. Detectors are in the form of one or more single element detectors or one or more arrays or bundles of detectors. Detectors include InGaAs, PbS, PbSe, Si, MCT, or the like. Detectors further include arrays of InGaAs, PbS, PbSe, Si, MCT, or the like. Light collection optics such as fiber optics, lenses, and mirrors are commonly used in various configurations within a spectrometer to direct light from the source to the detector by way of a sample.

Dynamic Properties of Skin

The dynamic properties of skin tissue is an important and largely ignored aspect of noninvasive glucose concentration determination. At a given measurement site, skin tissue is often assumed to remain static, except for changes in the target analyte concentration and the concentration of other interfering species. However, variations in the physiological state and fluid distribution of tissue profoundly affect the optical properties of tissue layers and compartments over a relatively short period of time.

Many factors impact the physical and chemical state of skin. These include environmental and physiological factors. A long list of such factors may be generated, but includes at least body temperature, environmental temperature, food intake, drug or medicine intake, and applied pressure to a sampling site. An impact on one part of the body affects many other locations in the body. For example, food intake into the digestive track results in movement of water between internal compartments. Another example is caffeine or stimulant intake changing blood pressure or dilation of capillaries.

### Noninvasive Glucose Concentration Determination

There exist a number of reports on noninvasive glucose technologies. Some of these relate to general instrumentation configurations required for noninvasive glucose concentration determination. Others refer to sampling technologies. Those most related to the present invention are briefly reviewed here:

As outlined above, there have been a number of studies documenting the need for an accurate and precise noninvasive glucose analyzer.

R. Barnes, J. Brasch, D. Purdy, W. Lougheed, Non-invasive determination of analyte concentration in body of mammals, U.S. Pat. No. 5,379,764 (Jan. 10, 1995) describe a noninvasive glucose concentration determination analyzer that uses data pretreatment in conjunction with a multivariate analysis to determine blood glucose concentrations.

General Instrumentation

P. Rolfe, Investigating substances in a patient's bloodstream, UK Patent Application No. 2,033,575 (Aug. 24, 1979) describe an apparatus for directing light into the body, detecting attenuated backscattered light, and utilizing the collected signal to determine glucose concentrations in or near the bloodstream. C. Dahne, D. Gross, Spectrophotometric method and apparatus for the non-invasive, U.S. Pat. No. 4,655,225 (Apr. 7, 1987) describe a method and apparatus for directing light into a patient's body, collecting transmitted or backscattered light, and determining glucose from selected near-IR wavelength bands. Wavelengths include 1560 to 1590, 1750 to 1780, 2085 to 2115, and 2255 to 2285 nm with at least one additional reference signal from 1000 to 2700 nm.

M. Robinson, K. Ward, R. Eaton, D. Haaland, Method and apparatus for determining the similarity of a biological analyte from a model constructed from known biological fluids, U.S. Pat. No. 4,975,581 (Dec. 4, 1990) describe a method and apparatus for measuring a concentration of a biological analyte such as glucose using infrared spectroscopy in conjunction with a multivariate model. The multivariate model is constructed form plural known biological fluid samples.

J. Hall, T. Cadell, Method and device for measuring concentration levels of blood constituents non-invasively, U.S. Pat. No. 5,361,758 (Nov. 8, 1994) describe a noninvasive device and method for determining analyte concentrations within a living subject utilizing polychromatic light, a wavelength separation device, and an array detector. The apparatus uses a receptor shaped to accept a fingertip with means for blocking extraneous light.

S. Malin, G Khalil, Method and apparatus for multi-spectral analysis of organic blood analytes in noninvasive infrared spectroscopy, U.S. Pat. No. 6,040,578 (Mar. 21, 2000) describe a method and apparatus for determination of an organic blood analyte using multi-spectral analysis in the near-IR. A plurality distinct nonoverlapping regions of wavelengths are incident upon a sample surface, diffusely reflected radiation is collected, and the analyte concentration is determined via chemometric techniques.

Temperature

K. Hazen, Glucose Determination in Biological Matrices Using Near-Infrared Spectroscopy, doctoral dissertation, University of Iowa (1995) describe the adverse effect of temperature on near-IR based glucose concentration determinations. Physiological constituents have near-IR absorbance spectra that are sensitive, in terms of magnitude and location, to localized temperature and the sensitivity impacts noninvasive glucose determination.

Guide

T. Blank, G. Acosta, M. Mattu, S. Monfre, Fiber optic probe guide placement guide, U.S. Pat. No. 6,415,167 (Jul. 2, 2002) describe a coupling fluid of one or more perfluoro compounds where a quantity of the coupling fluid is placed at an interface of the optical probe and measurement site. Perfluoro compounds do not have the toxicity associated with chlorofluorocarbons. Blank also teaches the use of a guide in conjunction with a noninvasive glucose analyzer to increase precision of the location of the sampled site resulting in increased accuracy and precision in a noninvasive glucose concentration determination. The guide is used for a period of time to increase precision in sampling throughout a period of sampling, such as a fraction of a day, one day, or a period of multiple days.

Mean Centering

E. Thomas and R. Rowe, Methods and apparatus for tailoring spectroscopic calibration models, U.S. Pat. No. 6,157,041, (Dec. 5, 2000) and E. Thomas and R. Rowe, Methods and apparatus for tailoring spectroscopic calibration models, U.S. Pat. No. 6,528,809, (Mar. 4, 2003) describe mean used in combination with a noninvasive glucose concentration analyzer. The guide embodiments are optionally used as an alternative approach to mean centering.

Equilibration

A number of reports exist describing the difference (or lack of difference) between traditional glucose concentration determinations and alternative site glucose concentration determinations. Some have recognized the potential difference as having impacts upon noninvasive glucose calibration and maintenance.

Differences between traditional and alternative site glucose concentrations have been presented in U.S. patent application Ser. No. 10/377,916, which is herein incorporated in its entirety by this reference thereto.

In-light Solutions (formerly Rio Grande Medical Technologies), has reported the use of heat, rubrifractants, or the application of topical pharmacologic or vasodilating agents such as nicotinic acid, methyl nicotinamide, minoxidil, nitroglycerin, histamine, menthol, capsaicin, and mixtures thereof to hasten the equilibration of the glucose concentration in the blood vessels with that of the interstitial fluid. See M. Rohrscheib, C. Gardner, M. Robinson, Method and Apparatus for Non-invasive blood analyte measurement with Fluid Compartment Equilibration, U.S. Pat. No. 6,240,306 (May 29, 2001) and M. Robinson, R. Messerschmidt, Method for Non-Invasive Blood Analyte Measurement with Improved Optical Interface, U.S. Pat. No. 6,152,876 (Nov. 28, 2000).

Nitric Oxide

Nitric oxide (NO) has been used to cause vasodilation. Nitric oxide is a free radical gas that behaves as an endogenous vasodilator which is important in regulation of circulation. Nitric oxide initiates and maintains vasodilation through a cascade of biological events that culminate in the relaxation of smooth muscle cells that line arteries, veins, and lymphatics. See R. Furchgott, Nitric Oxide: From Basic Research on Isolated Blood Vessels to Clinical Relevance in Diabetes”, An R Acad Nac Med (Madrid), 115, 317-331 (1998). While somewhat complex, the sequence of biological events that are triggered by NO is outlined below:
Step 1. NO gas released from nitrosothiols in hemoglobin or from endothelial cells, diffuses into smooth muscle cells that line small blood vessels.
Step 2.Once inside the smooth muscle cell, NO binds to an enzyme, called guanylate cyclase (GC) and this binding results in GC activation.
Step 3. Activated GC is able to cleave two phosphate groups from another compound called guanosine triphosphate (GTP). This results in the formation of cyclic guanosine monophosphate (cGMP) that is used to phosphorylate (Phosphorylation is the addition of a phosphate group) proteins, including the smooth muscle contractile protein called myosin.
Step 4. Once phosphorylated, smooth muscle cell myosin relaxes, resulting in dilation of the vessel that was originally exposed to NO.

Essentially, nitric oxide is a signaling molecule that is known to relax smooth muscle in arteries, veins, and lymph vessels. When these vessel muscles relax they dilate, which results in increased circulation through decreased resistance. See D. Carnegie, The Use of Monochromatic Infrared Energy Therapy in Podiatry, Podiatry Management, 129-134 (November/December 2002).

### Photo Stimulation

Nitric oxide is stored in cells such as red blood cells. Dr. R. F. Furchgott noted that nitric oxide could be acutely released when white light is presented to tissues resulting in increased blood flow. Because light is made up of several different wavelengths, subsequent research studies explored the beneficial effects of individual wavelength to determine which might be better at causing NO production or release thus stimulating vasodilation. Studies with visible colors were followed by experiments with monochromatic sources of non-visible light such as ultraviolet and near-infrared. The Anodyne Therapy System™ uses near-infrared light to accomplish the local release of NO from hemoglobin and possibly other heme proteins within red blood cells. The Anodyne Therapy System™ uses monochromatic light at 890 nm to stimulate the NO release (see Carnegie, supra.) and other devices have been reported using 890 nm light stimulation. See G. Noble, A. Lowe, D. Baxter, Monochromatic Infrared Irradiation (890 nm): Effect of a Multisource Array upon Conduction in the Human Median Nerve, J. of Clin. Laser Medicine and Surgery, 19, 291-295 (2001).

Release of nitric oxide via photo stimulation has been suggested for such uses as pain mediation, wound healing and tissue repair, and to increase circulation with implications to medical treatment of circulatory related problems associated with ulcers, eyes, kidneys, heart, and the intestine. However, this technology has not been suggested for use in combination with noninvasive glucose concentration determination. See D. Bertwell, J. Markham, Photo-thermal Therapeutic Device and Method, U.S. Pat. No. 5,358,503 (Oct. 25, 1994). Further, minimization of reference glucose concentration differences has not been suggested with the use of photo stimulation. Finally, to date no FDA device has been approved for use by an individual or a medical professional for noninvasive glucose concentration determination.

### The Problem

The body is dynamic in nature. Body constituents are subject to input and output events that occur at non-uniform times and in fashions that are not equally distributed through the body. This results in certain body constituents constantly being in a state of flux. For example, the glucose concentration in the body is not equally distributed in different body compartments. Even within the circulatory system, glucose is not always evenly distributed. Difficulties arise when one portion of the body is sampled to determine or measure a constituent concentration when it is desirable to determine the concentration of that constituent in an alternative body part. An example is glucose measured at an alternative site such as the forearm when it is desirable to determine the fingertip, arterial, or venous glucose concentration. This invention provides a method and apparatus for enhancing perfusion of capillary, tissue, or skin layers such that the concentration of analytes in the sampled region is used to more accurately or precisely determine the analyte concentration in other body parts that are less accessible in terms of required technologies, time, money, convenience, or pain.

### SUMMARY OF THE INVENTION

A method and apparatus using photo-stimulation to treat or pretreat a sample site prior to analyte concentration determination is presented. More particularly, photo-stimulation at or near at least one sample site is used to enhance perfusion of the sample site leading to reduced errors associated with sampling. Increased perfusion of the sample site leads to increased volume percentages of the target analyte and/or allows the blood or tissue constituent concentrations to more accurately and/or precisely track corresponding sample constituents in more well perfused body compartments or sites such as arteries, veins, or fingertips. In one embodiment, analysis of the photo-stimulated site is used in conjunction with glucose analyzers to determine the analyte concentration with greater ease, accuracy, or precision and allows determination of the analyte concentration of another non-sampled body part or compartment.

### BRIEF DESCRIPTION OF THE DRAWINGS

FIG. 1 is a graph that shows a dampening and lag in forearm glucose concentration profile versus a fingertip reference profile;

FIG. 2 is a graph that shows improved correlation in glucose concentration profiles between a photo-stimulated site and a reference fingertip sample site compared to a non-photostimulated sample site, according to the invention;

FIG. 3 is a graph that shows that noninvasive glucose concentration determinations performed at photo-stimulated sites predict with increased accuracy the capillary blood glucose concentration versus alternative site blood glucose concentration, according to the invention;

FIG. 4 is a graph that shows noninvasive glucose concentration predictions from photostimulated sites versus capillary glucose reference concentrations, according to the invention;

FIG. 5 is a graph that shows predictions from an untreated site using a noninvasive glucose concentration analyzer versus a fingertip reference glucose concentration for six subjects;

FIG. 6 is a graph that shows predictions from a treated site using a noninvasive glucose concentration analyzer versus a fingertip reference glucose concentration for six subjects, according to the invention;

FIG. 7 is a schematic diagram that shows an LED plug attachment coupled to a guide, according to the invention;

FIG. 8 is a schematic diagram that shows an LED attachment coupled to a plug with a 4.5 inch radius of curvature guide, according to the invention; and

FIG. 9 is a schematic diagram that shows a miniaturized source attachment coupled to a 6.0 inch radius of curvature guide, according to the invention.

### DETAILED DESCRIPTION OF THE INVENTION

The invention comprises a method that uses photo-stimulation in conjunction with the relative or absolute concentration determination of body analytes. More particularly, photo-stimulation at or near at least one sample site is used to enhance perfusion of the sample site such that the blood or tissue constituent concentrations more accurately and/or precisely track corresponding sample constituents in the more well perfused body compartments or sites, such as arteries, veins, or fingertips. Means for determining the analyte concentration at or near the photo-stimulated site then observe a sample or location with analyte concentrations that are more representative of the well perfused regions of the body. Means for determining the analyte concentration include invasive, minimally invasive, or noninvasive methods. The analyte concentration determining means include direct and indirect methods. Methods and apparatus for determining the analyte concentration of interest include impedance, chromatographic, electrochemical, or spectroscopic means. The analyte includes any constituent of blood or of an analyte that tracks the concentration of a blood constituent. One particular analyte of interest is glucose. Other sample constituents of interest include fats such as triglycerides or forms of cholesterol, proteins such as albumin or globulin, urea, bilirubin, and electrolytes such as Na⁺, Ca²⁺, and K⁺ or various chelates.

Analyte Distribution

Constituents of blood that are acquired from outside sources, generated, or consumed are not equally distributed in the body. For example, it is well known that the oxygen concentration of arterial blood is greater after the lungs compared with the oxygen concentration of venous blood returning to the lungs. Still lower oxygen concentrations are found in poorly perfused regions of the body. Generally, analytes that are picked up or dropped off by blood have different concentrations in different portions of the body at the same time due to the localized rate of change of the constituent being faster than the replenishing or equalizing circulatory flow at less well perfused sites. For example, the concentration of a blood constituent near the skin surface may differ from that of the concentration of the same analyte in the well perfused regions of the circulatory system. Concentrations of analytes in interstitial fluid are also dependent upon the perfusion of nearby regions. For example, the concentration of glucose in interstitial fluid decreases with time due to glycolysis.

The decrease in glucose concentration is dependent upon both distance from a capillary bed and the history of perfusion of the capillary bed. Hence, as the perfusion of nearby regions is enhanced, it affects both capillary and interstitial glucose concentrations. Often, it is desirable to measure or determine the general concentration of such an analyte in the body while sampling at a localized site is preferred.

Glucose Distribution

Glucose is concentrated in aqueous based body compartments. Further, within aqueous body compartments, glucose is not evenly distributed. Certainly intracellular and extracellular glucose concentrations differ. In addition, intra-vascular glucose concentration is different in different parts of the body at the same time. Generally, the circulatory system moves blood glucose rapidly through the main arterial/venous channels. In well perfused capillary beds, such as the fingertips, the glucose concentration is roughly equivalent to that of the main arterial and venous compartments. Generally speaking, the concentration of glucose is uniform in the main arterial/venous circulatory system, though some glucose is consumed by the body such that arterial glucose concentration may exceed those of venous glucose concentrations. Again, some glucose is used in the capillary regions that decreases the localized glucose concentration but as the perfusion rates are large the glucose concentrations do not vary considerably. However, some regions of the body are not as well perfused as that of the fingertip. Generally speaking, less well circulated or perfused regions are more likely to have periods in which the glucose concentration differs from the more well perfused regions of the body.

Additional differences result from glucose metabolism and synthesis. Again, it is often desirable to measure or determine the general concentration of glucose in the body with a test at a localized site. One method of increasing the localized perfusion so as to obtain a more representative sample is photo-stimulation described herein.

A detailed description of glucose differences between traditional invasive sites such as the fingertip and alternative invasive sites such as the forearm has been previously provided in U.S. application Ser. No. 10/377,916 (filed Feb. 28, 2003), which is herein incorporated in it is entirety by this reference thereto. Some key points are summarized here.

Differences between traditional invasive and alternative invasive glucose determinations are demonstrated. It is demonstrated here that the differences between the alternative invasive glucose concentration from a site such as the forearm and the glucose concentration from a traditional invasive fingerstick vary as a function of at least time and location. Additional parameters include sampling methodology, physiology, and glucose analyzer instrumentation.

For example, variation of glucose concentration at locations in the body is demonstrated. A diabetic subject was run through a glucose perturbation. Over a period of four hours the glucose concentration started low at around 80 mg/dL, was increased to circa 350 mg/dL, and was brought back to circa 80 mg/dL. This profile was generated with intake of approximately 75 g of a liquid form of carbohydrate in combination with subsequent injection of insulin to generate the ‘N’ profile. Traditional invasive fingertip capillary glucose concentrations were determined every 15 minutes through the four-hour protocol and were followed as quickly in time as the operator could obtain with alternative invasive capillary glucose determinations with samples collected from the volar aspect of a given subject's right and then left forearm. This resulted in 69 data points.

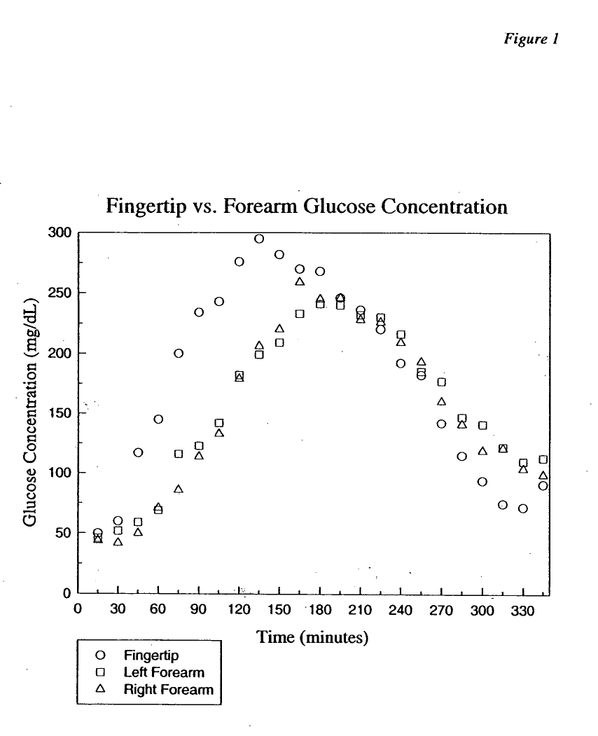

The resulting glucose concentration profile is presented in FIG. 1. The alternative invasive glucose concentrations measured at the forearm are demonstrated to be substantially dampened, lower than the corresponding fingertip glucose concentration. The alternative invasive glucose concentrations are also observed to have a lagged profile versus the traditional invasive fingertip glucose concentrations.

Several conclusions are drawn from this and previously presented data. First, during a glucose excursion, substantial differences are sometimes observed between the capillary blood glucose of the untreated forearm and the fingertip. Second, rapid changes in blood glucose concentration magnify differences between the measured blood glucose concentration of the fingertip and forearm while the relative errors are proportional to the glucose concentration. Third, during periods of rapid change in blood glucose concentration, differences between the forearm and fingertip give rise to a higher percentage of points in less desirable regions of the Clarke error grid. Fourth, the measured blood glucose concentrations of the volar aspect of the left and right forearms appear similar. Finally, These findings are consistent with the mechanism of decreased perfusion into the forearm versus that of the fingertip leading to a dampening and/or lag in the glucose profile.

Physiology

The above listed conclusions are consistent with the circulatory physiology literature and sampling approaches of alternative invasive glucose analyzers. It has been reported that blood flow in the fingers is 33±10 ml/g/min at 20° C. while in the leg, forearm, and abdomen the blood flow is 4-6 mL/g/min at 19-22° C. This is consistent with the observed differences in localized blood glucose concentration. When glucose concentrations vary rapidly, a difference develops through-out the body in local blood glucose concentration as a result of differences in local tissue perfusion. For example, the blood flow in the fingers of the hand is greater than in alternative sites. This means that the blood glucose concentration in the fingertips equilibrates more rapidly with venous blood glucose concentrations. Furthermore, the magnitude of differences in local glucose concentrations between two sites is related to the rate of change in blood glucose concentration. Conversely, under steady-state glucose conditions, the glucose concentration through-out the body tends to be uniform.

The following physiological interpretations are deduced from these studies. First, during times of glucose change, the concentration on the arm can lag behind that of the fingertip. Second, a well-recognized difference between the fingertip and the forearm is the rate of blood flow. Third, differences in circulatory physiology of the off-finger test sites leads to differences in the blood glucose concentration. Fourth, on average, the arm and finger glucose concentrations are the same, but the correlation is not one-to-one. This suggests differences between traditional invasive glucose concentrations and alternative invasive glucose concentrations are different during time periods of fasting and after glucose ingestion. Fifth, the relationship of forearm and thigh glucose concentration to finger glucose concentration is affected by proximity to a meal. Meter forearm and thigh results during the 60 and 90 minute testing sessions are consistently lower than the corresponding finger results. Sixth, differences are inversely related to the direction of blood glucose change. Seventh, in some cases rapid changes produce significant differences in blood glucose concentrations measured at the fingertip and forearm. Eighth, for individuals, the relationship between forearm and finger blood glucose concentration may be consistent. However, the magnitude of the day-to-day differences has been found to vary. Finally, in some instances interstitial fluid (ISF) glucose concentrations lead, as oppose to lag, plasma glucose concentrations, such as in the case of falling glucose concentrations due to exercise or glucose uptake due to insulin. One method of increasing the localized perfusion is photo-stimulation described herein.

### Photo-stimulation

Photo-stimulation is also referred to as photostimulation, photonic stimulation, or stimulation or excitation with light or photons. Photostimulation is herein used to refer to photons being absorbed by an absorber that subsequently releases an agent that results in increased perfusion. Photostimulation is distinct from photonic heating. Photonic heating may be used in conjunction with photostimulation.

Photo-stimulation at or near the sample site is performed in a manner that enhances perfusion of the sample site primarily by enhancing or inducing perfusion of the sample site.

Photo Stimulation in Combination with Glucose Determination

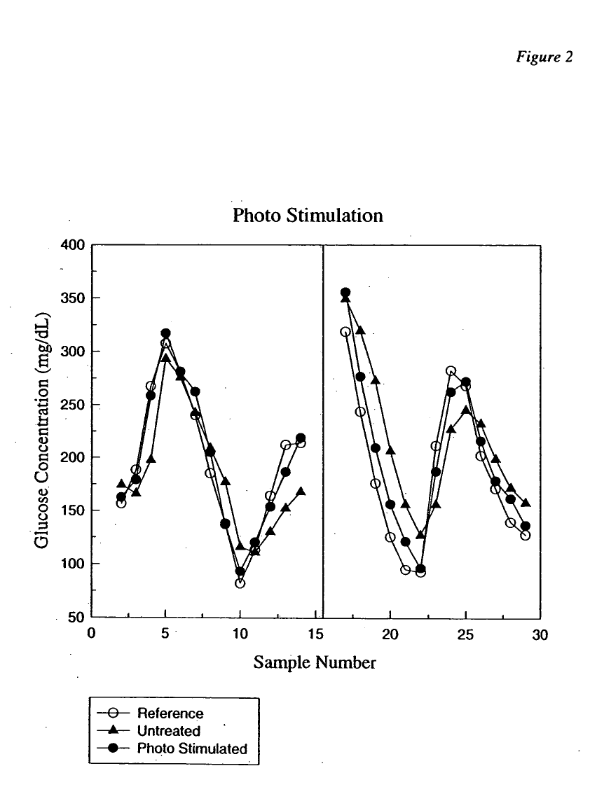

Photonic stimulation was used to reduce or eliminate the differences in the glucose concentration between the alternative sampling site of the forearm and the traditional sampling site of the fingertip in terms of dampening and lag. In one study, a number of subjects were run through glucose excursions driven by the combined use of carbohydrate intake and insulin injections. In this particular study, one forearm site was pretreated with 890 nm photostimulation while the contralateral site, on the opposite forearm, and fingertips were left untreated. The 890 nm stimulation was performed with three 890 nm LEDs for a period of 30 minutes immediately prior to the glucose concentration data collection. Invasive glucose concentration determinations were subsequently obtained every 20 minutes from all three locations. For two representative subjects, the resulting glucose concentration profiles are presented in FIG. 2. In the first case, the photo-stimulated site is observed to have a higher correlation with the fingertip reference glucose concentration compared to the untreated site. Both the dampening and lag observed in the untreated forearm versus the fingertip are not observed in the glucose profile obtained from the photostimulated site. This indicates that the photo-stimulated site is better perfused. In the second presented example, the dampening and lag of the photostimulated site is observed to be less pronounced than compared to the untreated site. However, some lag is still initially observed. Subsequently, better optical coupling techniques were used that reduced the percentage of subjects that showed a lag.

The increased perfusion that results in the alternative site glucose concentrations more closely tracking the traditional site (fingertip) glucose concentrations is important for several reasons. Medical professionals and diabetes educators have been trained for a generation on the treatment of diabetes with the use of arterial or fingertip glucose concentrations. A large body of literature and indeed medical practice is based upon traditional site glucose concentration determinations. A systematic difference between the body sites will lead to a systematic bias in treatment of diabetes by these educators until medical practice is altered. While the FDA has allowed manufacture, sale, and use of glucose concentration determination methods and apparatus for alternative site glucose concentration determination, they have separate labeling requirements in terms of testing during stable glucose periods and instruction not to rely on alternative site glucose determination for timely detection of hypoglycemia. The large number of glucose concentration equalization approaches by large companies such as heating, partial vacuum, and rubbing of the sample site as outlined above is further evidence of the importance of an equalization approach. Further, an error calculation of a medical device of a well perfused and/or equalized sample alternative sampling site versus a traditional site fingerstick reference will have better accuracy and precision compared to an untreated alternative site glucose error calculation versus a traditional fingertip reference method.

### Exemplary Apparatus for Photostimulation

A photonic stimulation device is either a stand alone device or is incorporated into a more complex apparatus. Various embodiments are described below where the photo-stimulation device is used alone (in the invasive glucose determination section) or as part of a larger device (in the noninvasive glucose determination section). These examples and the description in this section are merely examples of the general device that includes a power supply and a photonic source. A general overview of a photonic-stimulation source with some possible embodiments follows in this section.

Source

A photo-stimulation apparatus includes at least a power supply and a source. A wide number of sources are available to achieve stimulation. These include light emitting diodes (LEDs), broadband sources, lasers, diode lasers, and a supercontinuous source.

A preferred photostimulation source is an LED. The source should enhance perfusion at or near a sample site. As detailed above, stimulation at 890 or 910 nm results in release of nitric oxide. A broader wavelength range is optionally used to stimulate the same release. The literature shows that the excitation group of interest is a sulfylhydryl group. Additional literature indicates that absorbance of the light by deoxyhemoglobin that is coordinated with the heme group results in the release of nitric oxide. Therefore, the broader potential range of photonic stimulation includes all regions where hemoglobin or the sulfylhydrl groups absorb. Naturally, as the absorbance of the agents responsible for the release of nitric oxide decreases the efficiency of coupling the light into the release of nitric oxide decreases. Therefore, wavelengths near the peak absorbances of the coupling molecular structures are preferable. Photostimulation ranges therefore include wavelength ranges that are absorbed by deoxyhemoglobin such as regions about 890 or 910 nm are, 850 to 950 nm or less, and preferably 700 to 1000 nm. Broader ranges are used with decreasing photonic efficiency. Other molecular structures that upon photo-stimulation result in the enhanced perfusion of a sample site have their own specific preferable excitation ranges. It should be recognized that as the photonic stimulation process occurs, there is some ancillary heating due to the physical processes associated with absorbance. However, the photonic stimulation process described herein stimulates a secondary action beyond heating to induce enhanced perfusion. This distinguishes the process from heating of the sample site for increased perfusion as taught by others. See M. Rohrscheib, U.S. Pat. No. 6,152,876, supra and M. Rohrscheib, U.S. Pat. No. 6,240,306, supra. Broadband light is not a preferable method of performing photo-stimulation because many of the wavelengths of a blackbody source do not induce nitric oxide release. The additional wavelengths may heat the tissue and cause dilation of capillaries increasing perfusion. As discussed in an alternative embodiment, this heating is advantageous in many situations. However, the undue heating of the sample site has its costs. First, a large amount of broadband light is not inducing nitric oxide release. This makes the system less efficient. For example, a larger source and/or power supply is required. Second, it is well known that undue heating of the sample results in many near-IR absorbance bands to change in a nonlinear fashion, which complicates subsequent analysis.

In one embodiment of the invention, a broadband source is used with optical filters. The optical filters include one or more longpass, shortpass, or bandpass filters used to isolate one or more spectral regions. This allows one or more wavelength region of interest to penetrate into the sample. Broadband sources are relatively inexpensive. Thermal control of the incident radiative and conductive heat coupled to the sample is preferable.

Alternative sources include lasers and laser diodes. Typically these sources are used to deliver a greater flux of photons. This allows a more rapid stimulation and subsequently a more rapid increase in perfusion. However, these devices are typically larger and more expensive. Hence, lasers and laser diodes achieve the objectives of the invention, but LED's are preferred. An alternative source is a supercontinuous source, which is optionally created inside of a fiber. A supercontinuous source is utilized in continuous wave or pulsed mode.

Source configuration include individual elements, multiple elements, and an array. For example, in the preferred embodiment, a single 910 nm LED is used for photo-stimulation. If a shorter illumination time is desired, two or more LED's are used for excitation at the expense of greater power consumption. Three LED's are optionally placed into a guide element as discussed below. An array of LED's is utilized to further shorten the required illumination time or to enhance stimulation of a larger area. For example, a patch with an m by n array of LED's is used to cover a larger surface area of the sample where m and n are integers.

More than one range of wavelengths are optionally used in the illumination source. For example, two or more types of LED's are used in the source. This results in a wider wavelength range of incident photons or two or more bands of incident photons. If additional mechanisms are identified that lead to dilation of capillaries based upon more than one absorbance feature, it may be beneficial to excite both functional groups. In addition, broader coverage of a given absorbance band is achieved in terms of wavelengths.

A mixture of species of illumination elements may be used for photostimulation. For example, an LED may be used in combination with a broadband source.

Power Supply

A source supplying the incident photons requires a power supply. Generally, the power supply is AC or DC. Selection of the appropriate power supply is dependent upon the particular application and is obvious to those skilled in the art. Several illustrative examples follow. If the illuminator is to be portable, then a battery power supply is preferable. A small device is constructed that couples the power supply to the source. Such a device may be handheld or replaceably attached to the sample site through, for example, a guide. If a larger number of incident photons are desirable to for instance diminish the required illumination time period, an AC power supply may be coupled to the source.

Coupling Optics

In the simplest embodiment of this invention, the photons travel through air to reach the sampling site. Alternatively, coupling optics are used.

Those skilled in the art will recognize that systems that enhance photon transport or direction to a sample site are used to optimize the flux of photons at the sample site. Typical elements to achieve this include reflectors, lenses, diffusers, and fiber optics. For example, a back reflecting mirror is placed behind a tungsten halogen source to focus light onto the sample or a focusing optic isused in front of the source. In another example, light is transported to a sample site via a fiber optic. A diffuser may broaden a narrow optical beam to illuminate a broader sampling surface. Clearly, these and related optical elements are used in combination to provide the desired coupling of the source light to the sample site. Those skilled in the art will immediately recognize that coupling optics are used with any of the above described illumination sources.

Sample Interface

The interface of the photonic source to the sample is of particular concern. In one embodiment, photons are coupled to the sample through free space optics. Alternatively, the optics are placed in contact with the sample. There are advantages to each method.

Free space optics are here defined as incident photons that are traveling through a gas such as air upon entry into the sample site. Alternatively, coupling optics are used where the surface of the sample site is in contact with a solid or a liquid.

Important considerations of the sample interface are accuracy of the incident photons to the targeted sample site, precision of photons to the targeted sample site, temperature impacts on the sample site, and pressure impacts on the sample site. Some examples of coupling methods follow.

### EXAMPLE 1

Incident photons, such as those from an LED, are coupled into the skin through air. This has the benefit of not disturbing the sample site by application of pressure. This is beneficial for a noninvasive measurement. However, for an invasive measurement this pressure impact may be minimal. Coupling photons into skin through air is not the most efficient coupling method due to the index of refraction mismatch and the optical roughness of skin.

### EXAMPLE 2

Incident photons are coupled to a sample site via coupling optics such as a fiber optic, one or more lenses or flat optics, and/or a coupling fluid. The coupling optics are in direct contact with the skin. This means that the thermal effects of the coupling optics on the sample surface impact the sampling site temperature. This is tolerated or controlled. Similarly, the coupling optics apply at least some pressure to the sampling site that may disturb the sampling site. Again, this may be tolerated or controlled. Those skilled in the mechanical arts will immediately recognize control techniques such as use of thermally stable materials, thermally less or nonconductive materials, temperature controllers, or adjusting the mass in contact with the sample site to achieve desirable thermal control. Those skilled in the mechanical arts will immediately recognize techniques to control pressure effects such as adjusting mass, distributing pressure over an area, use of counter forces, or perturbing the sample known or preset distances.

Alignment

The accuracy and/or precision of the incident photons relative to the sampling site is important. For example, the increased perfusion due to the incident photons is limited in surface area and its associated volume. Generally, sampling in the perfused region is desirable. Embodiments where sampling outside of the perfused region is desirable are described in the alternative embodiments below. Many possibilities exist for sampling where the perfusion is enhanced; a few are described below.

One method of sampling where the perfusion is enhanced is by visually aligning sampling to be where the photons were incident upon the skin. This is performed in a number of ways such as memory, spatially relative to one or more sample features such as a joint or a freckle, or by measurement.

Another method of sampling where the perfusion is enhanced is by using a larger illumination area. For example, a diffusing optic or an array of illuminators is used.

A third method of sampling where perfusion is enhanced is by the use of a guide. Guides are described in detail below. Generally, a guide is a replaceably attached apparatus used as one-half of a lock and key mechanism. One use of a guide is the alignment of the incident photons relative to the sampling site and/or the alignment of a sensor or probing device relative to the same sampling site.

### Exemplary Method for Photostimulation

In the simplest embodiment of this invention, photostimulation is performed prior to and/or during sampling.

The relative timing of photostimulation and sampling is dependent upon the application. Specific examples of duty cycles and timing relative to sampling are provided in the preferred and alternative embodiments. Several illustrative examples of timing of stimulation follow.

### EXAMPLE 3

In some instances the photostimulator is not optically attached to the sample site when not in use. In these cases the source is manually turned on or is activated with automatic activation means known to those skilled in the art. For example, activation means include inducement by pressure applied when sampling, by a switch mechanism in a guide, by sensing movement, or by proximity to a magnetic field. Once activated, the duty cycle is continuous or semi-continuous. Photostimulation duration controls include manual and automatically deactivated after a preset time interval. Photostimulation periods include the beginning of a day or operating period, prior to sampling by multiple minutes, just prior to sampling, and during sampling.

### EXAMPLE 4

If the photostimulator is optically attached to the sample site, the duty cycle is continuous, semi-continuous, or manually activated by the user. For example, an LED photostimulator is in a guide element and the stimulator is programmed to turn on at a given time of day, continuously illuminate, have a duty cycle, or have manual activation means.

Permutations and combinations of methods and apparatus for photonic stimulation sources described in this section are used in conjunction with analyzers or incorporated into analyzers. As described above, the analyzers may analyze blood constituents or constituents that may be indirectly measured by the impacts of increased perfusion. Several specific illustrative embodiments are described below.

### Exemplary Embodiments

As discussed above, a preferred embodiment of the invention includes the use of photo-stimulation in conjunction with glucose sampling and/or measurement techniques. More particularly, photo-stimulation at or near a sample site is used to enhance perfusion of the sample site such that the blood or tissue concentration of glucose more accurately tracks that of arterial, venous, fingertip, or well perfused body site glucose concentration. Photostimulation, glucose sampling, and glucose concentration determination techniques are performed as described throughout this specification. The glucose concentration determinations are invasive, minimally invasive, or noninvasive. The invasive glucose determinations are preferably at alternative sites, but are optionally at a traditional site. Several embodiments of these species are described below.

One preferred embodiment of the invention is the use of photo-stimulation in conjunction with the noninvasive determination of glucose concentration. More particularly, photo-stimulation at or near a sample site is used to enhance perfusion of the sample site such that the blood or tissue concentration of glucose more accurately tracks that of arterial, venous, fingertip, or well perfused body site glucose concentration.

A wide range of noninvasive glucose concentration analyzers are known in the art. The spectrophotometric based noninvasive glucose analyzers include a source, light directing optics, a sample, detection means, and data analysis means. Permutations and combinations of photon based noninvasive analyzers are well known. U.S. Pat. No. 6,040,578 and U.S. application Ser. No. 10/366,085, PCT Application Number PCT/US03/07065, and U.S. Provisional No. 60/448,840 have been previously described and are herein incorporated in their entirety by reference.

A preferred embodiment uses photostimulation as outlined in this specification in combination with a noninvasive glucose analyzer as outlined herein. Particular embodiments are described here.

In a first embodiment of the invention, a photonic-stimulator is used in combination with a noninvasive glucose analyzer to generate glucose concentration determinations from at least one subject. The noninvasive analyzer includes a source, a sample, light direction optics, and at least one detector. The analyzer preprocesses the data and uses multivariate analysis in the glucose concentration determination.

In a second particular embodiment, a noninvasive glucose concentration analyzer is used in combination with photonic stimulation. The photonic stimulator is packaged in a plug that couples into a guide. Preferably, the applied pressure of the plug to the tissue sample is controlled through means such as a spring. The plug contains at least one LED or equivalent. Preferably, the LED is centered at approximately 890 or 910 nm and is battery powered. The LED is used to photo stimulate the sample site at least prior to the first glucose determination of a day. The glucose analyzer includes a tungsten halogen source, an optional backreflector, and at least one optical filter prior to the sample. The optical filter is used as a heat blocker and/or as an order sorter. The preferred embodiment directs the incident light onto a sample, preferably the back of the wrist about one inch toward the elbow from the wrist joint through the use of a guide. Photons are collected from the sample and are directed to a grating and subsequently to at least one detector. The spectral range is 1100 to 2500 nm or at least one range therein. Preprocessing is performed on the spectra. Forms of at least one of averaging, smoothing, taking the nth derivative, clustering, performing multivariate analysis and mean centering are performed. A glucose concentration is generated. In this preferred embodiment, the absorbance of glucose is the key analytical signal, though an indirect method is optionally used as the analytical signal as described in U.S. application Ser. No. 10/349,573, (filed Jan. 22, 2003), which is herein incorporated herein in its entirety by this reference thereto.

### EXAMPLE 5

In another particular embodiment, a noninvasive glucose analyzer is used in combination with photonic stimulation. The photonic stimulator is packaged in a plug that couples into a guide. The plug contains a single element 890 nm LED run off of a battery that is used to photostimulate the sample site at least prior to the first glucose determination of a day. The glucose analyzer includes a tungsten halogen source of less than five Watts, a back reflector, and at least two optical filters prior to the sample. At least one of the optical filters is used as a heat blocker and as an order sorter. The sample module preferably applies minimal pressure to the sample site. The preferred embodiment directs the incident light onto a sample, preferably the back of the wrist through the use of a guide. Diffusely reflected photons are collected from the sample into at least one fiber optic and are directed to a grating and subsequently to an array detector. The spectral range is 1150 to 1800 nm or ranges therein. Preprocessing is performed on the spectra. Forms of at least one of averaging, smoothing, taking the nth derivative, clustering, performing multivariate analysis and mean centering are performed. A glucose concentration is generated.

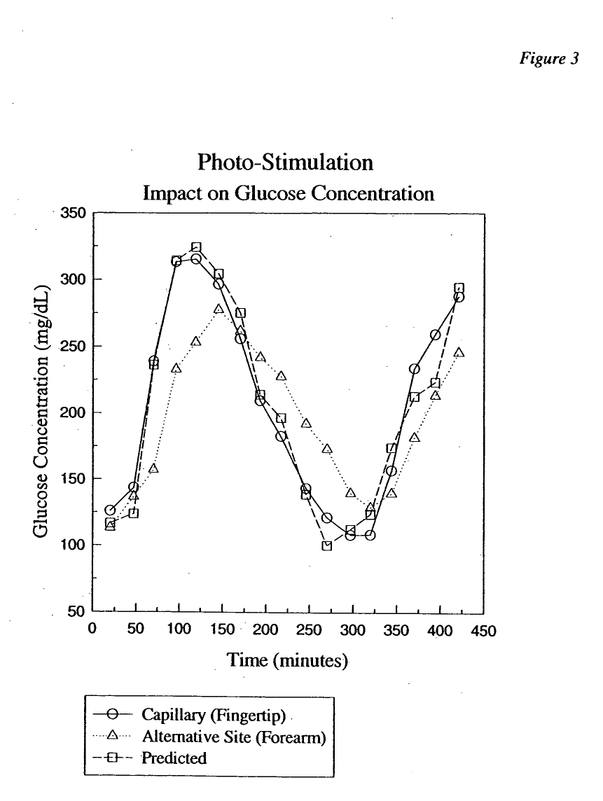

Glucose concentration predictions using the noninvasive glucose apparatus of the embodiment described in the last paragraph are presented in FIG. 3. This glucose concentration profile is that of a single subject. The glucose rises were induced through carbohydrate intake to create a large glucose concentration test range. Insulin was used to bring the glucose concentrations down to test the predictive power of the model on the glucose signal instead of an ancillary correlation. Carbohydrates were subsequently ingested to test further the model by breaking remaining correlations between glucose and ancillary interferences. A noninvasive glucose concentration determinations was performed approximately every 20 to 25 minutes as were traditional fingertip glucose concentration determination and alternative site glucose concentration determination from a site on the forearm that was not treated. Clearly, the noninvasive glucose concentration predictions track the reference glucose concentrations. Of note, the predicted glucose concentrations from the photostimulated site track the fingertip reference glucose concentrations more accurately and precisely than the alternative site forearm reference glucose concentrations.

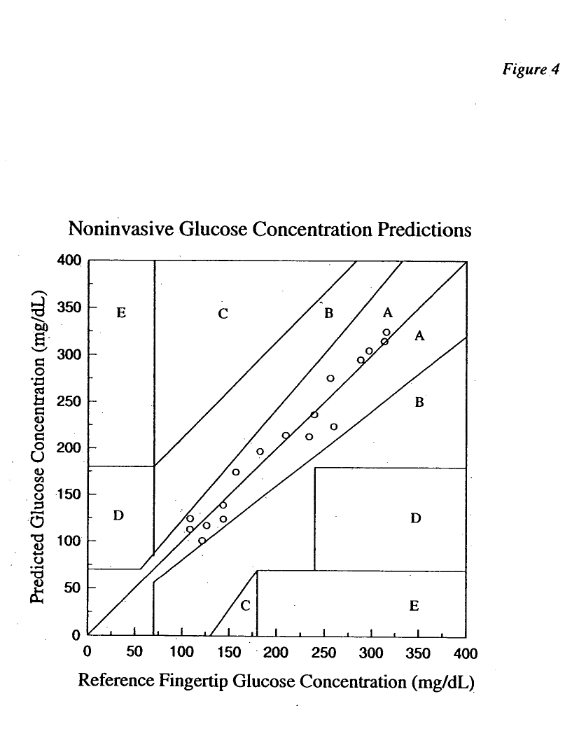

The noninvasive glucose concentration predictions and the fingertip reference glucose concentrations from FIG. 3 are plotted in a concentration correlation plot overlaid with a traditional Clarke error grid in FIG. 4. In a Clarke error grid, all points in the ‘A’ and ‘B’ region are clinically acceptable with the points in the ‘A’ region having less than 20 percent error. A crude guide to acceptable data is 95% of the points falling into the ‘A’ or ‘B’ region. In this study, 100% of the values fell into the ‘A’ region. The standard error of prediction is 14.6 mg/dL, the r is 0.98, and the F-value is 27.17.

### EXAMPLE 6

Another example of noninvasive glucose concentration predictions is provided with and without photonic stimulation. Sensys Medical pilot glucose analyzers were used in this study. The pilot analyzers included a tungsten halogen source, a back reflector, a silicon window, a guide, a plug fit into the guide, a sample (forearm), a single fiber optic was used to collected diffusely reflected light, a slit, a grating, and an array of detectors. Critical to the analyzer is the resulting signal to noise level, stability, and resolution of the analyzer as opposed to the specific elements used.

The guide was configured with a photonic stimulator attachment. In this case, three 890 nm LED's were used in the guide and were positioned roughly 1 mm from the sample site surface. A total of six subjects participated in this study. Each subject was treated with photonic stimulation on one arm over the sampling site and not on the opposite arm for a period of 30 minutes prior to collection of any noninvasive glucose spectra on a given test day. In this example, photostimulation was performed only prior to the first noninvasive glucose concentration determination and was not repeated prior to subsequent noninvasive or invasive glucose concentration determinations. Each subject was then run through a glucose excursion lasting for approximately four hours. Reference glucose concentration determinations were collected every 20 minutes from the fingertip and forearm with an invasive glucose concentration analyzer. In addition, noninvasive spectra were collected every 20 minutes from each forearm representing samples from untreated and photonically treated sample sites. One-half of the subjects were treated with photonic stimulation on their left arm and one-half were treated on their right arm.

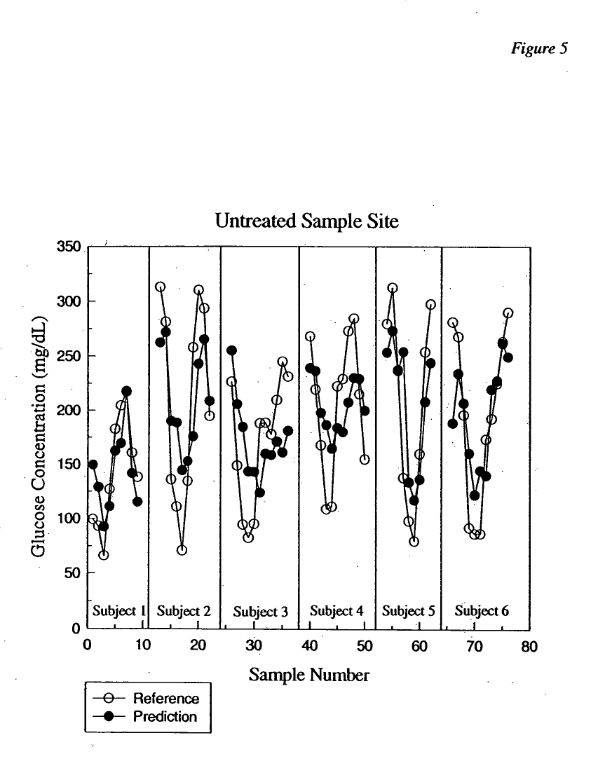

For each of the six subjects, the noninvasive spectra were analyzed with a calibration model. The model included a spectral preprocessing routine, an outlier analysis module, and a multivariate analysis module. The spectral range was 1200 to 1800 nm. The resulting glucose predictions from the noninvasive spectra collected from the untreated sample site of each of the six individuals is overlaid with their corresponding invasive reference glucose concentration determinations, FIG. 5. For subjects 2 through 5, the predicted glucose concentrations using the noninvasive analyzer clearly are dampened in their total glucose range relative to the reference glucose concentrations. Subjects 1 to 4 and subject 6 clearly have a predicted glucose profile that lags the reference glucose concentration. This is consistent with having a glucose concentration at the sampling site that is not well perfused and results in a glucose profile that is dampened and/or lagged versus a well perfused reference glucose region such as a fingertip.

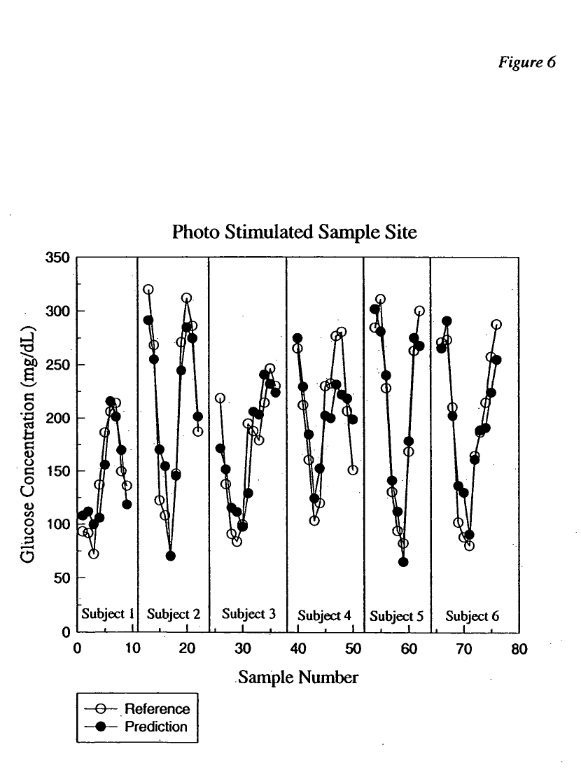

The resulting glucose concentration predictions from the noninvasive spectra collected from the treated sample site of each of the six individuals is overlaid with their corresponding invasive reference glucose determinations, FIG. 6. For subjects 1 to 3, 5 and 6, the predicted glucose concentration using the noninvasive analyzer closely tracks the reference glucose concentrations. Subject 4 has a predicted glucose concentration profile that initially tracks and later is dampened versus their corresponding reference glucose concentrations. These results are consistent with the photo stimulation treatment of the sampling site equalizing the glucose concentration between the fingertip and the forearm sample site. Further, the equalization persisted in all but one of the subjects over the entire 4 hour test period.

Photo stimulation is observed in the above study to result in equilibration of the glucose concentration between the less well perfused sample site and the well perfused reference site. Again, the photo stimulation results in vasodilation that led to the equilibration of the glucose concentration in the two body compartments. The noninvasive glucose concentration model was then able to predict more accurately the glucose concentration due to the noninvasive analyzer sampling a region that actually had glucose concentrations that correlate with the reference glucose concentration.

In the above study, photo stimulation was performed for 30 minutes with three LED's at the beginning of a daily testing period. The resulting vasodilation resulted in increased perfusion of the sampling site for a period of hours. Additional data indicates that a single LED results in the same vasodilation results. Therefore, one LED is sufficient to equalize the glucose concentration to the extent that a noninvasive glucose analyzer predicts more accurate glucose concentrations. Optionally, photo stimulation is performed periodically throughout a given testing period, such as a day, rather than just at the beginning of a day. For example, photo stimulation is used before the first sample of a day, with each sample of the day, or at periodic intervals during the day. The duration of stimulation of each interval is for either a fixed or variable time period. For example, the first photo stimulation duration of the day is preferably longer than subsequent treatments of the sample site.

### Alternative Embodiments

An alternative embodiment of the invention includes the use of photostimulation in combination with alternative invasive or even traditional invasive glucose concentration determination.

Preferably, the invention includes the use of photo-stimulation to induce perfusion in combination with sampling and/or measurement techniques of alternative invasive glucose determination. However, the technique is beneficial for traditional sampling sites in subjects such as diabetics that have poor circulation in their extremities.

As outlined in the background section, a number of approaches are used to attempt to equalize the glucose concentration at alternative sampling sites prior to analysis with alternative invasive glucose analyzers. These pre-sampling techniques have included heating, rubbing, and pulling negative pressures. The invention herein includes the substitution of photostimulation for any of these techniques.

Photostimulation as described throughout this specification includes use in combination with alternative invasive glucose sampling and determination techniques. Some specific embodiments follow. Note that these specific embodiments are intended to be species of the genus and are illustrative of the larger technique.

### EXAMPLE 7

A handheld photostimulator is used in conjunction with alternative invasive sampling and/or analysis techniques.

Photostimulation sources for the handheld device are as described elsewhere in this specification. For example, one or more 890 nm LEDs is powered by a battery to provide photons that are delivered to the sample where they are subsequently absorbed leading to increased perfusion of the sample site. The power supply, source, and optional coupling optics are integrated into a handheld illuminator. The device optionally includes a means for turning the device on or off. The device is used to photostimulate prior to and/or during an alternative invasive glucose determination.

In alternative embodiments of a handheld device, other photostimulation sources are used as described throughout this document. For example sources include one or more LEDs, broadband sources, broadband sources coupled with longpass, shortpass, or bandpass optics, lasers, and diode lasers.

### EXAMPLE 8

An invasive glucose determination is combined with the use of photostimulation. A photostimulator is incorporated into a guide or made as one-half of a lock and key guide mechanism. For example, one or more 910 nm LEDs is incorporated into a plug along with one or more batteries. The plug is replaceably attached to a guide. The guide itself is replaceably attached to or near the sampling site.

The photostimulator is an option described in the embodiments where the photostimulator is coupled to a noninvasive glucose analyzer. For example, the photostimulator is configured to run continuously, be activated by a user, to have preset duty cycles, be motion activated, or be activated by means such as a magnetic field when placed near the sampling site.

Considerations of the photostimulator apparatus and method of use include power consumption, size, cost, stability, accuracy of alignment to the sample site, precision of alignment to the sample site, and lifetime.

As described above, the photostimulator optionally has one or more source elements or an array of sources. A photostimulation apparatus is coupled to the sample site with free space, floating, or fixed coupling optics.

Photostimulation is performed at or near the sampling site. Therefore, if photostimulation is performed at a different time period from when sampling is performed it is important to have locating means such that sampling occurs at or near the photostimulation site. Means described in the noninvasive embodiments would be applicable here. For example, locating means include direct measurement, memory, distances to sample features, or relative distances to sample features are used. In addition, a replaceably attached guide may be used as described below. It should be noted that in the case of an invasive or semi-invasive glucose determination, the guide need not be left on the sampling site for extended periods of time. It is sufficient to place a guide, photostimulate in a position relative to the guide, sample in a position stimulated and remove the guide. Typically, in a noninvasive glucose determination the guide would be left on for a series of glucose concentration determinations.

Preferable sampling sites include the forearm, wrist area, upper arm, torso, thigh, and ear. Photostimulation is optionally used prior to traditional glucose analysis on locations such as the fingertip, base of thumb, plantar regions, or toes. This is beneficial for diabetics with circulation problems where traditional sampling of blood is difficult. The increased perfusion allows for smaller lancets and shorter penetration depths for adequate blood volume to be collected and/or used.

As in the noninvasive embodiment, photostimulation is used prior to each sampling period, continuously, with a duty cycle, in a manually controlled fashion, with a set timer, or by automatically activation by proximity to a site.

The use of photostimulation in combination with invasive glucose concentration determination methods has a number of advantages. First, the combination allows for more accurate and precise glucose concentration determinations when compared to traditional fingertip glucose concentration determinations. Second, the decreased lag time makes invasive meters more useful in determination of hypoglycemia. Third, the decrease in dampening allows for more accurate determinations of glucose extremes during hyperglycemic periods. Fourth, photostimulation allows glucose concentration analysis while glucose concentrations are changing rapidly, for example in excess of 2 mg/dL/min.

Heating

Photostimulation is intended to replace equilibration techniques described herein such as heating, rubbing, and pulling partial vacuums. However, it is recognized that there are benefits of using photostimulation in combination with these techniques.

Photo-stimulation is used in conjunction with heating to enhance perfusion of the sample site. The combined perfusion enhancement is then followed by noninvasive or alternative invasive techniques as described in the preferred embodiments above.

Several sources have taught the benefits of heating the sampling site prior to glucose analysis for both noninvasive and alternative site sampling methodologies. Some benefits of heating the sampling site include dilation of the capillaries to enhance localized circulation and stabilization of the temperature of the sampling site to minimize spectral variation. Heating used in combination with photonic stimulation results in the benefits of photonic stimulation and heating. Heating may be radiative or conductive. For example, a heating element placed in close proximity to the sampled site provides heating. This heating element is optionally controlled with a feedback sensor. See K. Hazen, Noninvasive Glucose Determination in Biological Matrices, Ph.D. dissertation, University of Iowa, Department of Chemistry (1995). Further, the heating is performed via absorbance of photons. As described infra, different wavelength light is used to preferentially heat different layers of the sample site due to the penetration depth of the photons as a function of wavelength.

Many sources are used to provide photonic heating including broadband radiative sources, broadband sources limited by filters to one or more spectral regions, glowbars, LEDs, laser diodes, and lasers. For example, a tungsten halogen source is coupled with one or more longpass, shortpass, or bandpass filters to pass light to the sample site with one or more regions.

It is possible to heat different tissue layers preferably via absorbance. This may result in the expansion of capillaries due to heat at preferable sampling depths without the interferences associated with undue heating at other sample depths. This is possible as some wavelengths penetrate further into the body based upon the scattering and absorbance coefficients of the illuminated site. Therefore, appropriate selection of wavelengths of incident light preferentially absorb and thus heat different skin depths with different efficiency. For example, mid-infrared (2500 to 14,258 nm or 4000 to 700 cm⁻¹) light absorbs in the first few microns of the skin surface due to the strong absorbance of water in these wavelength ranges. Combination band light (2000 to 2500 nm) preferentially absorbs in skin resulting in heat at a greater depth of circa 1-2 mm. First overtone (1450 to 1950), second overtone (1100 to 1450), preferentially absorbs at depths of 1 to 5 and 4 to 10 mm of depth, respectively due to the absorbance of water. Therapeutic window light penetrates and heats at greater depths, but is highly influenced by the scattering properties of the sample. Visible light is highly scattered and results in heating at a large range of depths. Selection of an appropriate range or ranges of wavelengths can result in preferential heating at one or more depths.

Differential Measurements

In an alternative embodiment, differential measurements in terms of photostimulation are performed. More particularly, temporal and/or spatial differential measurements are performed. Differential measurements are often made in spectroscopy to enhance a signal to noise level or determine a difference in state.

A temporal differential measurement is made by performing an analysis before, during, and/or after photostimulation. Typically, a baseline reading is performed. For example, a noninvasive spectrum is obtained. Photostimulation is then performed. A second noninvasive spectrum is then obtained. Many chemometric approaches then use the two spectra. Typically, these techniques are subtraction or ratio determination to remove background information or enhance the analyte signal to noise level. For example, the signal to noise level of glucose, oxygen, or urea are enhanced. Alternatively, differential measurements are used to determine the impact of photostimulation on the sample site.

A spatial differential measurement is made by performing an analysis at two sites. One site is treated by photostimulation and the other site is left untreated. Typically, both analyses are performed at the same time or close in time such as within a few seconds or minutes. For example, a baseline reading is performed at the untreated site and a sampling reading is performed at the treated site. For example, in spectroscopy the reference spectrum is collected at the untreated site and the sample spectrum is collected at the treated site. Typically these spectra are subtracted from one another or ratioed to enhance the signal to noise level of an analyte, though additional chemometric approaches may be used. For example, the signal to noise level of glucose, oxygenation levels, or urea may be enhanced.

Alternative Analyte Determinations

In an alternative embodiment, photostimulation is used in combination with noninvasive urea, cholesterol, blood gas, oxygen, or pH determination. Noninvasive determinations of urea concentration and pH have been disclosed in the literature. Noninvasive techniques used for glucose concentration determination that are described herein and in the literature may be used. Wavelength regions for urea, blood gases, cholesterol, and pH have been disclosed. See U.S. Pat. Nos. 6,212,424; 5,630,413; 5,792,050; 6,061,581; and 6,073,037.

### Lock and Key Elements

In many embodiments of this invention, a guide is used. The following discussion describes guides, guide placement, and guide use. The embodiments of the lock and key (guide) mechanisms and methods described herein are applicable to above embodiments.

Lock (Guide)

A guide may be replaceably attached to a sample site. The guide is one-half of a lock and key mechanism. That is attachments are replaceably attached to the guide or inserted into the guide. A number of guide (lock) configurations exist and a number of attachments (keys) exist. Many of these have been previously described in U.S. Pat. No. 6,415,167; U.S. patent application Ser. No. 10/170,921; and provisional application No. 60/472,613, which are all herein incorporated in their entirety by reference. The photostimulation apparatus embodiments described herein attach to any of the aforementioned guide elements. In addition, several related guide configurations are described herein.

It has been determined that matching the shape of the guide to the structure of the sample site results in increased precision of subsequent optical sampling. For example, an arm sampling site varies between individuals in terms of circumference or radius of curvature. For the case of an arm sampling site, the skinnier the arm the smaller the radius of curvature of the optimal guide. Guides have been used that have a flat, 6.0 inch, 4.5 inch, and 3.0 inch radius of curvature. At the sample site, the guide surface may be flat. Thus, one embodiment of a guide is to have the surface interfacing with a sample such as a forearm to have the shape of the outside sides of a cylinder that has been modified to be flat near the sample site by a plane.

A core feature of the guide element is that is makes up one-half of a lock and key combination. That is a surface exists that reproducibly guides the other half of a lock and key element into a reproducible position. In this case, the lock element is in the guide, but alternatively it is in the attachment. In this case, the lock element is a hole in the guide that is roughly rectangular with two opposing sides each having rounded shapes. The rectangular shape limits rotational alignment. Preferably, the guide would not have rotational freedom. For instance the pictured guide may be rotated by 180 degrees. This rotational freedom could be eliminated by flattening one of the round ends. Many lock element shapes are readily used. Examples include virtually any geometrically shaped hole or any shape (not necessarily a hole) that provides reproducible positioning while preferably preventing freedom of rotation. In the particular guide elements presented, optional additional holes or divots are pictured. The function of these is primarily to reduce weight, minimize surface abnormalities such as sink marks on the sampling site, and to maintain strength while limiting the twisting freedom of the guide. An additional optional component pictured on these guides are magnets. The magnets are used to control contact force and/or to aid in alignment of the lock and key mechanism. In the guide pictured, optional opposing pole magnets are also placed into the plug. Of the paired magnets, one half of the pair could be a be metallic substance such as sheet metal or stainless steel. This may be done to reduce cost and/or weight.

The guide is attached to the sampling site with a number of means including a band, a strap, Velcro, or preferentially with a double sided adhesive. Commonly the adhesive is firmly placed onto the sampling site and then the guide is visually aligned onto the adhesive. This sequence reduces separation events of the adhesive from the sampling site. Optionally, the adhesive is attached to the guide and the pair are placed into contact with the sampling site as a unit. This eases alignment of the guide to the adhesive. The guide and adhesive are semi-permanently and removeably attached to the sampling site. The guide is typically left in place for the remainder of a sampling period such as one waking day or the length of a data collection period such as four or eight hours.

An optional intermediate layer or guide extension is used between the guide and the double sided adhesive that attaches to the sampling site. Essentially, this is a semi-flexible material such as acetate. The material allows some flexibility to allow the sample site skin to stretch. This reduces sampling transients resulting from movement of the subject. Conversely, in subjects with poor turgor, the skin flexes too much and a more rigid insert such as a plastic film is optionally used.

The guide is preferentially formed out of a thermoplastic such as a polycarbonate or a polyurethane. However, many materials will be obvious to those skilled in the art. Since the guide is in contact with the sampling site (sometimes with an intermediate adhesive), the thermal properties of the guide become important. Typically, the guide is non-thermally conductive to reduce sampling site temperature gradients. However, in some cases a thermally conductive guide is preferential such as when heat flow to or from the sample site is desired. The guide material should be biocompatible.

The guide is optionally optically coupled to the sampling site through the use of an index of refraction matching medium such as a fluoropolymer, a fluorocompound, Fluorinert, FC-40, FC-70, or equivalent.

Key (Attachment)

The other half of the guide lock and key mechanism is herein referred to as an attachment to the guide element. Several attachments including a plug, photonic stimulator, and miniaturized source have been previously described. A key feature of each of the attachments is that they each have the second half of the lock and key mechanism used in conjunction with the guide element. Again, this aids in reproducible positioning of the attachment in relation to the guide. Notably, any curvature guide may be used with any of the four attachments.

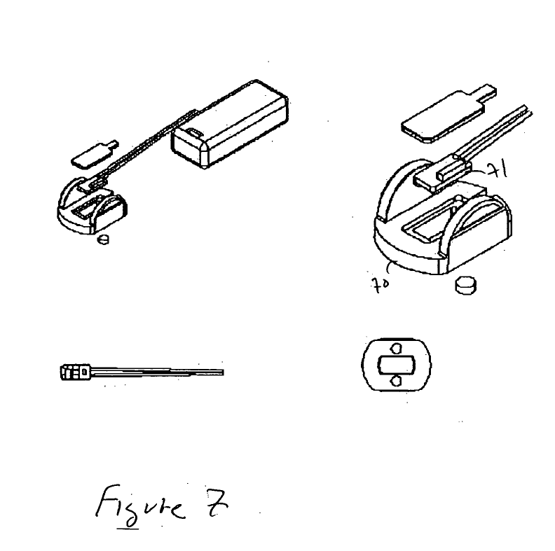

An additional embodiment of a photonic stimulator placed into a guide is provided here. A photonic stimulator attachment is presented in FIG. 7 coupled to a guide. In the embodiment pictured, a guide 70 is coupled to a plug 71. The plug contains three LEDs along with a circuit board. Power is supplied via an auxiliary battery or power pack. The power supply may be integrated into the plug. In this example, magnets are used to facilitate reproducible alignment between the guide and the plug and hence between the plug containing the LEDs and the sample site.

The photonic stimulator attachment results in many of the advantages or properties of a plug. The photonic stimulator attachment is optionally used as a plug to accomplish at least one of hydration of the sampling site by occlusion, protection of the sampling site from physical perturbation, protection of the sampling site from contamination, alignment of the guide, and allowing an aesthetic appearance such as a watch, ring, or graphical symbol.

The primary function of the photonic stimulator, however, is to increase localized perfusion as described throughout this specification. The difference in glucose concentration in different body compartments and the importance of this difference to noninvasive glucose calibration, maintenance, and prediction is presented in detail in U.S. patent application Ser. No. 10/377,916, which is herein incorporated in its entirety this by reference thereto.

In the preferred embodiment, the photonic stimulator is incorporated into an attachment that fits into a guide, as shown. In an alternative embodiment, the LED's are automatically turned on when the attachment is placed into the guide. In this case, the copper insert in the guide completes a contact with the metal pins of the attachment. A battery is placed into the photonic stimulator guide. Optionally, the attachment is configured with a button or switch to manually power on/off the source. Optionally, the power to the LED's runs from a base module to the attachment as described below for the miniaturized source attachment.

In another embodiment, there exists a commonality of the lock and key mechanism of the various guides and the various attachments for quick interchange and reproducible placement of; for example the plug, photonic stimulator, and the miniaturized source relative to the sample site. Further it allows the photonic stimulator or miniaturized source attachment to be rapidly and reproducibly aligned relative to the reference guide.

Photostimulation for Glucose Equalization

A number of optional elements are incorporated into the sampling module and/or guide to increase sampling precision and to increase the net analyte signal for the indirect glucose determination. These optional elements are preferably powered through the base module and connection cable described below but are alternatively battery operated. Equalization approaches include photonic stimulation, ultrasound pretreatment, mechanical stimulation, and heating. Notably, equilibration of the glucose concentration between the sampled site and a well-perfused region such as an artery or the capillary bed of the fingertip is not required. A minimization of the difference in glucose concentration between the two regions aids in subsequent glucose concentration determination.

The guide optionally contains an LED providing photonic stimulation about 890 nm, which is known to induce capillary blood vessel dilation. This technique is used to aid in equilibration of alternative site glucose concentrations with those of capillary blood glucose concentrations. By increasing the vessel dilation, and thereby the blood flow rate to the alternate site, the limiting nature of mass transfer rates and their effect on blood glucose concentration differences in tissue is minimized. The resulting effect is to reduce the differences between the finger and the alternate site blood glucose concentrations. The preferred embodiment uses (nominally) 890 nm LED's in an array set into the arm guide. Control electronics are embedded into the arm guide are remote. The LED's can also be used in a continuous monitoring application where they are located in the probe sensing tip at the tissue interface. Due to the periods of excitation required for stimulation, the 890 nm LED is preferably powered by a rechargeable battery in the guide so that the LED has power when the communication bundle is not used.

The guide optionally contains an apparatus capable of delivering ultrasound energy into the sample site. Again, this technique is used to aid in equilibration of alternative site glucose concentrations with capillary blood glucose concentrations by stimulating perfusion and/or blood flow.

The guide optionally contains an apparatus that provides mechanical stimulation of the sampled site prior to spectral data acquisition. One example is a piezoelectric modulator than pulses in an out relative to the skin surface a distance of circa 5 to 50 μm in a continuous or duty cycle fashion.

The guide optionally contains a heating and/or cooling element, such as a strip heater or an energy transfer pad. Heating is one mechanism of glucose compartment equilibration. These elements are used to match a target temperature, to manipulate the local perfusion of blood, to avoid sweating and/or to modify the distribution of fluids among the various tissue compartments.

### EXAMPLE 9

Photonic stimulation is investigated for the effects on the glucose concentration at a preferred sampling site versus that of a traditional fingerstick glucose determination. The objective of this study is to reduce or eliminate lag between the capillary based fingertip glucose concentration and the glucose concentration at a forearm measurement site.

For each subject tested in this study, glucose measurements using an invasive stick meter were obtained from two contralateral forearm sites and from a traditional fingertip site every 20 minutes during a glucose excursion. One forearm site was pretreated with 890 nm photo-stimulation. The photo-stimulated site is observed to have a higher correlation with the fingertip reference glucose concentration compared to the untreated site. The photo-stimulated site has less lag and less dampening than the untreated site. This indicates that the photo-stimulated site is better perfused.

The photonic stimulator preferably uses light about 890 nm as an FDA approved device has been approved using that wavelength for photo stimulation. However, the approved device is based upon a monochromatic wavelength stimulation. As the reported mechanism is initiated with the light energy being absorbed by hemoglobin, a wider range of photon wavelengths produce the same effect. For instance, available wavelengths of excitation include wavelengths that hemoglobin absorbs.

Within the near-IR (700 to 2500 nm), varying wavelengths absorb in the body primarily due to water at different levels. Therefore, wavelength selection could be used that focused the light at different depths within the tissue. For example, from 1100 to 1300, 1500 to 1800, 2100 to 2300, and 1400 to 1450 nm the light penetrates approximately 10, 3, 1, and 0.5 mm into water, respectively. One or more wavelengths within these regions is used. The use of multiple wavelengths is alternatively achieve with a broadband source in combination with a longpass, shortpass, or bandpass filter.

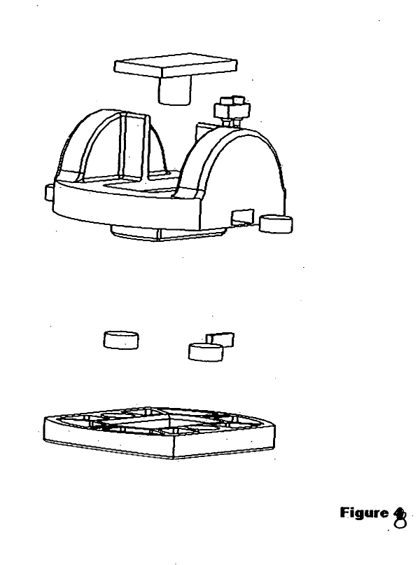

In this embodiment, the photonic stimulator is incorporated into an attachment that fits into a guide, as shown in FIG. 8. In the device picture, the LED's are automatically turned on when the attachment is placed into the guide. In this case, the copper insert in the guide completes a contact with the metal pins of the attachment. A battery is placed into the photonic stimulator guide. Optionally, the attachment is configured with a button or switch to manually power on/off the source. Optionally, the power to the LED's is provided by a base module to the attachment as described below for the miniaturized source attachment.

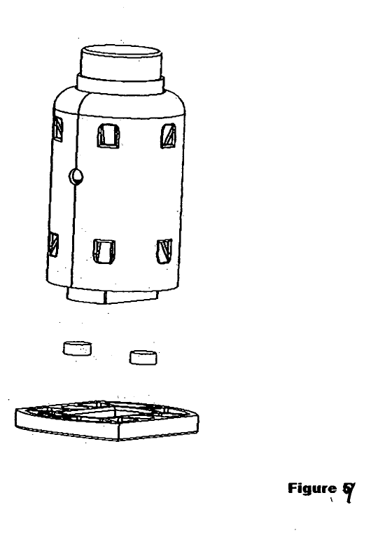

In an alternative embodiment, a miniaturized source attachment is presented in FIG. 9 coupled to a guide. The miniaturize source attachment results in many of the advantages or properties of a plug. The miniaturized source attachment is used as a plug to accomplish at least one of hydration of the sampling site by occlusion, protection of the sampling site from physical perturbation, protection of the sampling site from contamination, alignment of the guide, and allowing an aesthetic appearance such as a watch, ring, or graphical symbol. However, the primary function of the miniaturized source attachment is to provide the source element and guiding optics to and/or from the skin of a noninvasive glucose analyzer. The miniaturized source attachment coupled to a guide as part of a noninvasive glucose analyzer has been extensively described in PCT application number US03/07065 (attorney docket number SENS001 1), which is herein incorporated in its entirety by reference. Alternatively, the power supply through a communication bundle from the sampling module to the base module is used to power a photonic stimulation source. Optionally, the miniaturized source attachment is used for photonic stimulation.

A reference guide is optionally attached at some points in time to the miniaturized light source attachment described above. In this configuration, the lock and key aspect of the guide/attachment is used to optically align a reference material relative to the miniaturized source. The commonality of the lock and key mechanism of the various guides and the various attachments describe above allows for quick interchange and reproducible placement of the plug, photonic stimulator, and the miniaturized source relative to the sample site. Further the design allows the photonic stimulator or miniaturized source attachment to be rapidly and reproducibly aligned relative to the reference guide. This allows, for example, the miniaturized source to be rapidly moved from a reference to the sample site. As those skilled in the art will recognize, this is important for collecting reference (wavelength and/or intensity) spectra for purposes such as conversion of single beam intensity spectra to absorbance and for maintaining or transferring calibrations.

In an alternative embodiment, photo-stimulation is used to enhance invasive glucose concentration determination. More particularly, photo-stimulation as herein described is used to increase localized blood flow in alternative site body compartments such as the forearm, upper arm, thigh, and skin.

Invasive and semi-invasive glucose determinations performed on alternative site locations often result in glucose concentrations that differ from the traditional fingerstick glucose concentrations. For example, if a subject ingests carbohydrates their glucose concentration first increases and then decreases as a function of time. The observed glucose concentration profile that initially increases and subsequently decreases at an alternative site is often dampened and/or lagged versus the traditional fingerstick glucose concentration determination. The use of photo-stimulation on the alternative site prior to the invasive or semi-invasive determination allows the region to be perfused with blood that more closely resembles the blood circulating in the arteries, veins, and traditional measurement sites such as the fingertip. The change of the state of the sampled area allows the invasive or minimally invasive technique to operate on tissue and/or blood that more closely resembles the traditional sampling sites. Therefore, the observed glucose concentrations more closely track those of traditional glucose determinations. In the above listed example, the dampening and/or lag is reduced.

Notably, photo-stimulation alters the state of the tissue/blood at or near the photo-stimulated volume. Therefore, blood constituents such as proteins, fats, ions, urea, and glucose will all track more closely the actual concentrations of the body. Hence, photo-stimulation affects sampling techniques related to other blood/tissue sampling techniques. A particular example is noninvasive urea determination.

Although the invention is described herein with reference to the preferred embodiment, one skilled in the art will readily appreciate that other applications may be substituted for those set forth herein without departing from the spirit and scope of the present invention. Accordingly, the invention should only be limited by the Claims included below.

1. A method for concentration determination of body analytes, comprising the steps of:
using photo-stimulation at or near at least one sample site to enhance perfusion of said sample site, wherein sample constituents more accurately and/or precisely track corresponding blood or tissue constituent concentrations in more well perfused body regions; and
determining analyte concentration based upon measurements made at or near said photo-stimulated site.

2. The method of claim 1, wherein said sample site comprises any of a person's forearm, wrist area, upper arm, torso, thigh, and ear.

3. The method of claim 1, wherein said determining step is any of invasive, minimally invasive, and noninvasive.

4. The method of claim 1, wherein said determining step is any of direct and indirect.

5. The method of claim 1, wherein said determining step is based upon any of impedance, chromatographic, electrochemical, and spectroscopic techniques.

6. The method of claim 1, wherein said analyte comprises any of a constituent of blood or of an analyte that tracks the concentration of a blood constituent.

7. The method of claim 1, wherein said analyte comprises any of glucose, fats such as triglycerides or forms of cholesterol, proteins such as albumin or globulin, urea, bilirubin, and electrolytes such as Na⁺, Ca²⁺, and K⁺ or various chelates.

8. An apparatus for photonic stimulation at or near a sample site pursuant to concentration determination of body analytes, comprising:
a photonic source for enhancing perfusion at or near said sample site, wherein sample constituents more accurately and/or precisely track corresponding blood or tissue constituent concentrations in more well perfused body regions; and
means for determining analyte concentration based upon measurements made at or near said photo-stimulated site.

9. The apparatus of claim 8, said photonic source comprising any of one or more LEDs, broadband sources, lasers, diode lasers, and supercontinuous sources.

10. The apparatus of claim 8, wherein said source provides stimulation at about any of 890 and 910 nm.

11. The apparatus of claim 8, wherein said source provides stimulation at a wavelength near a peak absorbance of coupling molecular structures.

12. The apparatus of claim 8, wherein said source stimulates a secondary action beyond heating to induce enhanced perfusion.

13. The apparatus of claim 8, wherein said source comprises:
a broadband source used with at least one optical filter;
wherein said optical filter comprises one or more longpass, shortpass, or bandpass filters that isolate one or more spectral regions.

14. The apparatus of claim 8, wherein said source is configured as any of an individual element, as multiple elements, or as an array.

15. The apparatus of claim 8, wherein said source provides more than one range of wavelengths.

16. The apparatus of claim 8, wherein said source comprises:
a mixture of species of illumination elements

17. The apparatus of claim 8, further comprising:
one or more coupling optics for optimizing the flux of photons at said sample site, said coupling optics comprising alone or in combination any of reflectors, lenses, diffusers, and fiber optics.

18. The apparatus of claim 8, further comprising:
an interface between said photonic source to said sample site.

19. The apparatus of claim 18, said interface comprising any of:
free space optics and coupling optics.

20. The apparatus of claim 8, further comprising:
a guide, comprising a replaceably attached apparatus used as one-half of a lock and key mechanism, for alignment of incident photons from said photonic source relative to said sampling site and/or alignment of a sensor or probing device relative to said sampling site.

21. The apparatus of claim 8, wherein said photonic source is optically attached to said sample site, and wherein said photonic source duty cycle is any of continuous, semi-continuous, or manually activated by a user.

22. An apparatus for photo-stimulation in conjunction with glucose sampling and/or measurement techniques, comprising:
a photonic source for photo-stimulation at or near a sample site to enhance perfusion of said sample site, wherein blood or tissue concentration of glucose more accurately tracks that of any of arterial, venous, fingertip, or well perfused body site glucose concentration; and
means for glucose determination by any of an invasive and a noninvasive technique.

23. The apparatus of claim 22, said means for glucose determination comprising:
a noninvasive analyzer, comprising a source, a sample, light direction optics, at least one detector, means for preprocessing data, and means for using multivariate analysis for glucose concentration determination.

24. The apparatus of claim 22, said photonic source comprising:
a photonic stimulator packaged in a plug that couples into a guide.

25. The apparatus of claim 24, said plug comprises at least one 890 nm LED run off of a battery and that is used to photostimulate said sample site at least prior to a first glucose determination of a day.

26. The apparatus of claim 23, said noninvasive analyzer comprising:
a tungsten halogen source;
an optional backreflector; and
at least one optical filter prior to said sample site, said optical filter used as a heat blocker and/or as an order sorter.

27. The apparatus of claim 22, said photonic source comprising:
a handheld photostimulator for use in conjunction with invasive sampling and/or analysis techniques.

28. The apparatus of claim 22, wherein said sampling sites comprises any of a forearm, wrist area, upper arm, torso, thigh, and ear.

29. The apparatus of claim 22, wherein photostimulation is used prior to traditional glucose concentration analysis and said traditional analysis is on locations which comprise any of a fingertip, base of thumb, plantar regions, or toes.

30. The apparatus of claim 22, further comprising:
a heater for use in conjunction with photo-stimulation to enhance perfusion of said sample site.

31. The apparatus of claim 30, said heater comprising any of:
broadband radiative sources, broadband sources limited by filters to one or more spectral regions, glowbars, LEDs, laser diodes, and lasers.

32. The apparatus of claim 31, said heater comprising:
a tungsten halogen source coupled with one or more longpass, shortpass, or bandpass filters to pass light to said sample site with one or more regions.

33. The apparatus of claim 31, said heater configured to heat different tissue layers via absorbance.

34. The apparatus of claim 22, said means for glucose determination comprising:
means for performing differential measurements comprising any of temporal and/or spatial differential measurements.

35. The apparatus of claim 34, wherein a temporal differential measurement is made by performing an analysis before, during, and/or after photostimulation.

36. The apparatus of claim 34, wherein differential measurements are used to determine the impact of photostimulation on said sample site.

37. The apparatus of claim 34, wherein a spatial differential measurement is made by performing an analysis at two sites, wherein a first site is treated by photostimulation and a second site is left untreated, wherein both analyses are performed at the same time or close in time.

38. An apparatus for photo-stimulation in conjunction with analyte sampling and/or measurement techniques, comprising:
a photonic source for photo-stimulation at or near a sample site to enhance perfusion of said sample site, wherein blood or tissue concentration of said analyte more accurately tracks that of any of arterial, venous, fingertip, or well perfused body site analyte concentration; and
means for analyte determination.

39. The apparatus of claim 38, said means for analyte determination comprising:
means for any of noninvasive glucose, urea, cholesterol, blood gas, oxygen, or pH determination.

40. The apparatus of claim 38, further comprising:
a lock and key mechanism associated with said photonic source and replaceably attached to said sample site.

41. The apparatus of claim 40, wherein at least one of said lock and said key is profiled to the structure of said sample site.

42. The apparatus of claim 40, said lock and key mechanism comprising:
a guide coupled to a plug, wherein said plug comprises a plurality of LEDs.

43. The apparatus of claim 42, said lock and key mechanism comprising:
one or more magnets for effecting reproducible alignment between said guide and said plug.

44. The apparatus of claim 42, wherein said LED's are automatically turned on when said plug is placed into said guide.

45. The apparatus of claim 38, wherein said means for analyte determination comprises an invasive technique.

46. The apparatus of claim 38, wherein said means for analyte determination comprises a noninvasive technique.

47. The apparatus of claim 38, wherein said means for analyte determination comprises a minimally invasive technique.
